SEQUENCE LISTING (11 sequences)

SEQ ID NO: 1 - protein, 12 aa, Homo sapiens
RHYLNLVTRQRY

SEQ ID NO: 2 - protein, 11 aa, Homo sapiens
HYLNLVTRQRY

SEQ ID NO: 3 - protein, 10 aa, Homo sapiens
YLNLVTRQRY

SEQ ID NO: 4 - protein, 9 aa, Homo sapiens
LNLVTRQRY

SEQ ID NO: 5 - protein, 8 aa, Homo sapiens
NLVTRQRY

SEQ ID NO: 6 - protein, 13 aa, Homo sapiens
  MOD_RES (1)..(13): ACETYLATION
CRHYLNLVTRQRY

SEQ ID NO: 7 - DNA, 36 bp, Homo sapiens
cgccactacctcaacctggtcacccggcagcggtat

SEQ ID NO: 8 - DNA, 33 bp, Homo sapiens
cactacctcaacctggtcacccggcagcggtat

SEQ ID NO: 9 - DNA, 30 bp, Homo sapiens
tacctcaacctggtcacccggcagcggtat

SEQ ID NO: 10 - DNA, 27 bp, Homo sapiens
ctcaacctggtcacccggcagcggtat

SEQ ID NO: 11 - DNA, 24 bp, Homo sapiens
aacctggtcacccggcagcggtat

(19) United States
(12) Patent Application Publication
(10) Pub. No.: US 2009/0105122 A1
(43) Pub. Date: Apr. 23, 2009

(54) Selective neuropeptide y2 receptor agonists

(75) Inventors: Kevin Lumb, Guilford, CT (US); Lynn Decarr, North Haven, CT (US); Philip Coish, North Haven, CT (US); Stephen O'Connor, Guilford, CT (US)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER PHARMACEUTICALS CORPORATION, WEST HAVEN, CT (US)

(21) Appl. No.: 10/576,674

(22) PCT Filed: Nov. 23, 2004
(86) PCT No.: PCT/US2004/039216

§ 371 Date: Dec. 5, 2008

Related U.S. Application Data
(60) Provisional application No. 60/525,482, filed on Nov. 25, 2003.

(51) Int. Cl. A61K 38/28; C07K 7/08; C07K 7/06; A61K 38/08; A61K 38/10

(52) U.S. Cl. 514/4; 530/327; 530/328; 514/14; 514/15

(57) ABSTRACT

This invention provides peptides that act as selective NPY2 receptor agonists in vitro and are efficacious in vivo to reduce food intake. The invention is a peptide selected from a specific group of derivatized NPY-related peptides, or functional equivalents thereof. The invention is also directed to a method of treating a metabolic disease in a mammal comprising administering a therapeutically effective amount of the peptides to said mammal to reduce food intake and body weight.

This application claims benefit of U.S. Provisional Application Ser. No. 60/525,482, filed Nov. 25, 2003, the contents of which is incorporated herein by reference in its entirety.

### FIELD OF THE INVENTION

This invention relates to Neuropeptide Y2 (NPY2) receptor selective agonist peptides and the use of such peptides for therapeutic purposes. The peptides of the present invention are useful in reducing body weight and/or modulation of appetite or caloric intake, thereby providing a treatment option for those individuals afflicted with a metabolic disorder such as obesity, type 2 diabetes, eating disorders, insulin-resistance syndrome (Syndrome X), impaired glucose tolerance (IGT), dyslipidemia, and cardiovascular disorders.

### BACKGROUND OF THE RELATED ART

Obesity and associated disorders are common and very serious public health problems in the United States and throughout the world. Obesity is a recognized risk factor for hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia. Additionally, there is a strong association of obesity with non-insulin dependent diabetes mellitus (NIDDM), and more than 80% of NIDDM patients are obese. Therefore, obesity creates a high-risk medical burden on society and an effective treatment is essential.

Neuropeptide Y (NPY), peptide YY (PYY), and pancreatic peptide (PP) are members of the pancreatic peptide (PP) family characterized by a 36-amino acid sequence with a tyrosine amide at the carboxy-terminus and six conserved C-terminal amino acids. There are five known neuropeptide Y receptor subtypes (NPY1, NPY2, NPY4, NPY5, and NPY6) and these receptors are responsible for many diverse physiologic actions including feeding regulation, energy homeostasis, locomotion, seizure, thermoregulation, circadian rhythms, anxiety, cardiorespiratory function, nociception, and fertility. PYY(3-36), a major circulating form of PYY corresponding to residues 3-36 of PYY, interacts with at least three NPY receptor subtypes (NPY1, NPY2, and NPY5).

Peripheral administration of PYY was first reported to decrease appetite in 1993 (Okada, Endocrinology Suppl:180, 1993). Recently, PYY(3-36) was reported to inhibit appetite in rodents and humans (Batterham, Nature 418:650-654, 2002). However, the ability of PYY(3-36) to suppress feeding is not easily replicated (Tschop, Nature 430:165, 2004), suggesting that unmodified PYY(3-36) is not necessarily an effective therapeutic option for the treatment of obesity (Tschop, Nature 430:165, 2004). The finding that PYY(3-36) has no effect on feeding in mice deficient in the NPY2 receptor subtype, but inhibits feeding in wild-type litter mates, supports the hypothesis that PYY(3-36) modulates feeding through the NPY2 receptor. Central administration of PYY(3-36) results in a robust stimulation of feeding mediated through NPY1 and NPY5 receptors. Activation of the NPY1 receptor may result in vasoconstriction (Pedrazzini, Nature Med. 4:722-6, 1998), while activation of the NPY5 receptor has been implicated in hypertrophy of cardiomyocytes (Bell, J. Pharm. Exp. Ther. 303:581-591, 2002). Selectivity for the NPY2 receptor over the NPY1 and NPY5 receptors would provide the potential benefits of reducing side-effects and improved safety profile. This development of a potent, selective NPY2 receptor agonist would be desirable for the treatment of obesity. NPY2 receptor selective agonists may also reduce co-morbidities associated with obesity, type 2 diabetes, eating disorders, insulin-resistance syndrome (Syndrome X), impaired glucose tolerance (IGT), dyslipidemia, and cardiovascular disorders.

### SUMMARY OF THE INVENTION

This invention provides peptides that function as selective agonists of the NPY2 receptor and these peptides may be utilized for the treatment of diseases and conditions that can be ameliorated by agents having NPY2 receptor agonist activity. For example, but not by way of limitation, these peptides inhibit feeding and promote weight loss. The peptides of this invention are selective NPY2 receptor agonists, having greater potency at the NPY2 receptor than at NPY1 and NPY5 receptors.

The invention is also directed to a method of treating obesity and/or other diseases or conditions affected by the peptides of this invention, preferably effected by the NPY2 receptor agonist function of the peptides of this invention, in a mammal comprising administering a therapeutically effective amount of any of the peptides of the present invention such as peptides of Formulae (I), (II), (III), (IV), and (V), or any peptide active at NPY2 to said mammal.

The peptides of the present invention may also be utilized in the prevention and/or treatment of obesity-related disorders such as diabetes, Syndrome X, impaired glucose tolerance, atherosclerotic disease, hyperlipidemia, hypercholesteremia, low HDL levels, hypertension, cardiovascular disease (including atherosclerosis, coronary heart disease, coronary artery disease, and hypertension), cerebrovascular disease and peripheral vessel disease; and other conditions identified herein, or function otherwise as described later herein.

One aspect of the invention is a peptide selected from the group consisting peptides of Formulae (I), (II), (III), (IV), and (V), and PEGylated derivatives, and fragments, derivatives, and variants thereof that demonstrate at least one biological function that is substantially the same as the peptides of the described herein (collectively, “peptides of this invention”), including functional equivalents thereof.

Another embodiment of the invention is a polynucleotide (e.g., SEQ ID NO: 7-11) that encodes the peptides of the present invention, and the attendant vectors and host cells necessary to recombinantly express the peptides of this invention.

Antibodies and antibody fragments that selectively bind the peptides of this invention are also provided. Such antibodies are useful in detecting the peptides of this invention, and may be identified and made by procedures well known in the art. For example, a polyclonal N-terminal IgG antibody and a monoclonal C-terminal Fab antibody have been generated which recognize peptides of this invention.

### DESCRIPTION OF THE INVENTION

This invention provides peptides, and fragments, derivatives and variants thereof that demonstrate at least one biological function that is substantially the same as the peptides of Formulae (I), (II), (III), (IV), and (V) (collectively, peptides of this invention). The peptides of this invention function in vitro as selective NPY2 receptor agonists that demonstrate a functional response in terms of receptor activation equal to the endogenous ligands (e.g., NPY or PYY) and decrease food intake in vivo. The peptides of this invention provide an improvement over the natural ligands PYY and NPY in terms of receptor selectivity, and thereby potentially reduce food intake without inducing other undesired effects due to NPY1 and NPY5 receptor agonism. Thus, the peptides of this invention will decrease food intake and promote weight loss.

The naturally occurring NPY2 receptor agonists NPY and PYY are agonists for the NPY2, NPY1, and NPY5 receptors. It is desirable to develop selective NPY2 agonists that do not function or have minimal activity at the NPY1 or NPY5 receptors.

Selectivity against binding the NPY1 or NPY5 receptors can be imparted by deletion of N-terminal residues from PYY (Balasubramaniam, Peptide Res., 1:32-35, 1988). However, this is also accompanied with a marked decrease in affinity for the NPY2 receptor, and subsequent lack of NPY2 receptor agonism. Acetylation of peptide fragments of NPY results in a higher affinity for the NPY2 receptor, as shown previously for longer fragments of PYY (Murase, J. Biochem. 119:37-41, 1996). The present invention provides novel N-terminal modifications that confer a striking increase in NPY2 receptor affinity accompanied by the retention of selectivity against NPY1 and NPY5 receptors (e.g., peptides of Formulae (I), (II), (III), (IV), and (V)) and are suitable for functional derivatization to impart desired attributes such as improved pharmacodynamic properties.

The current state of the art for a beneficial N-terminal modification of a highly truncated peptide is acetylation of PYY(25-36) (e.g., Murase, J. Biochem. 119:37-41, 1996). While providing some improvement over the unmodified peptide in terms of NPY2 affinity, the acetylated peptide is not suitable for site-specific derivatization with a modification to improve efficacy in vivo, such as PEGylation or lipidation.

The present invention provides novel modifications that yield higher affinity and activity at the NPY2 receptor than the extant modification of acetylation while maintaining selectivity against the NPY1 and NPY5 receptors. Such N-terminal modifications at the amino group of the first peptide residue may include aliphatics, five- or six-member alkyls, five- or six-member heterocycles containing one or more nitrogen or sulfur heteroatoms. In addition, the N-terminal modifications may provide suitable derivatization sites (amino and thiol groups). Examples of such N-terminal modifications include, but are not limited to, 2-amino benzoic acid, 3-amino benzoic acid, 4-amino benzoic acid, 4-amino-2-chloro-benzoic acid, 4-amino-3-methoxy-benzoic acid, 4-amino-3-methyl-benzoic acid, 1-amino-cyclopentane-3-carboxylic acid, trans-3-aminocyclohexane carboxylic acid, D-pipecolinic acid, 4-amino-1-methyl-1H-imidazole-2-carboxylic acid, 4-methythiobenzoic acid, 2-methythiobenzoic acid, 2-methythionicotinic acid, proline, 6-aminohexanoic acid, benzoic acid, (S)-tetrahydroisoquinoline acetic acid, indoline-2-carboxylic acid, cis-3-aminocyclohexane carboxylic acid, L-pipecolinic acid, 9-gluorenylmethoxycarbonyl, 2-thio-polyethylene glycol (5 kDa) benzoic acid, 2-thio-polyethylene glycol (5 kDa) nicotinic acid, 4-amino-1-methyl-1H-imidazole-2-carboxylic acid, 1-amino-cyclopentane-3-carboxylic acid, 4-amino-1-methyl-1H-imidazole-2-carboxylic acid, 1-amino-cyclopentane-3-carboxylic acid, and 1-amino-cyclopentane-3-carboxylic acid (see, e.g., peptides of Formulae (I), (II), (III), (IV), and (V)).

It should be noted that enhancement in selectivity and NPY2 receptor affinity are surprisingly specific to the moiety attached to the N-terminus of the peptide. In particular, the novel modifications described herein provide higher affinity and activity at the NPY2 receptor than the extant modification of acetylation while maintaining selectivity against the NPY1 and NPY5 receptors. These findings indicate that the enhancement in NPY2 receptor affinity and selectivity are specific to the moiety attached to the N-terminus of the peptide, a result that would not be predicted a priori.

The effect of the modification also depends on the fragment of PYY. For example, while marked (yet not universal) effects of N-terminal modification are seen in the context of PYY(25-36), similar modification have benign effects in the context of PYY(22-26). N-terminal modifications do, however, improve the potency and selectivity of PYY fragments that are shorter than PYY(25-36). For example, but not by way of limitation, novel N-terminal modifications of PYY(26-36) and PYY(27-36) result in beneficial effects on NPY2 affinity and selectivity relative to acetylation. These finding are not a priori predictable to one skilled in the art.

Peptides may be derivatized to substantially preserve the functions disclosed and impart beneficial pharmacodynamic or other properties. For example, PEGylated peptides typically have greater half-life in vivo (Greenwald, Adv. Drug. Del. Rev. 55:217-250, 2003). The in vitro profiles of PEGylated N-terminally modified peptides demonstrate that PEGylation does not abrogate the in vitro properties of the peptides in terms of NPY2 binding or receptor selectivity.

The peptides of this invention are NPY2 receptor agonists. That is, the peptides of this invention are selective agonists (e.g., at least 5-fold selectivity) of the NPY2 receptor, thereby causing, for example, a decrease in food intake, while being selective against other receptors that are responsible for such undesired effects as appetite enhancement, and/or unwanted effects such as hypertension.

The disclosed peptides, derivatives, and modifications thereof, would be expected to decrease food intake and result in body weight loss in mammals. By way of example, but not of limitation, a peptide of the present invention reduces food intake in the fasted-refed lean mice model.

The invention relates to a peptide of Formula (I)

| | |
|---|---|
| Z-RHYLNLVTRQRY-NH₂ | (SEQ ID NO: 1) |
| (I) | |

wherein

Z is selected from

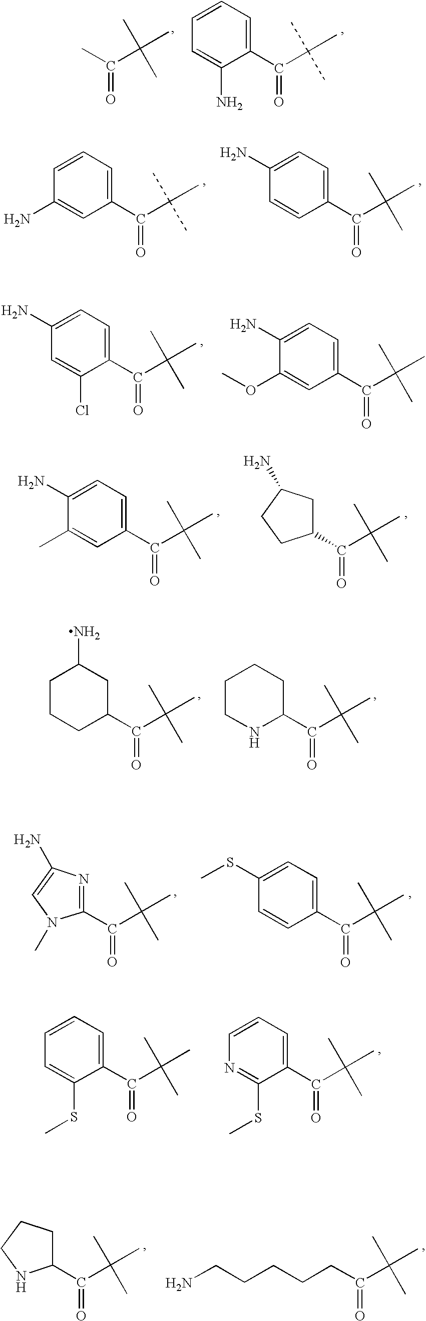

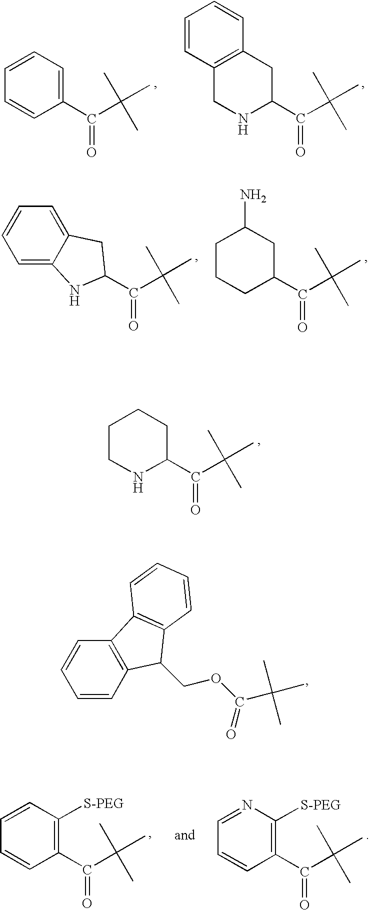

The invention relates to a peptide of Formula (II)

| | |
|---|---|
| Z-HYLNLVTRQRY-NH₂ | (SEQ ID NO:2) |
| (II) | |

wherein

Z is selected from

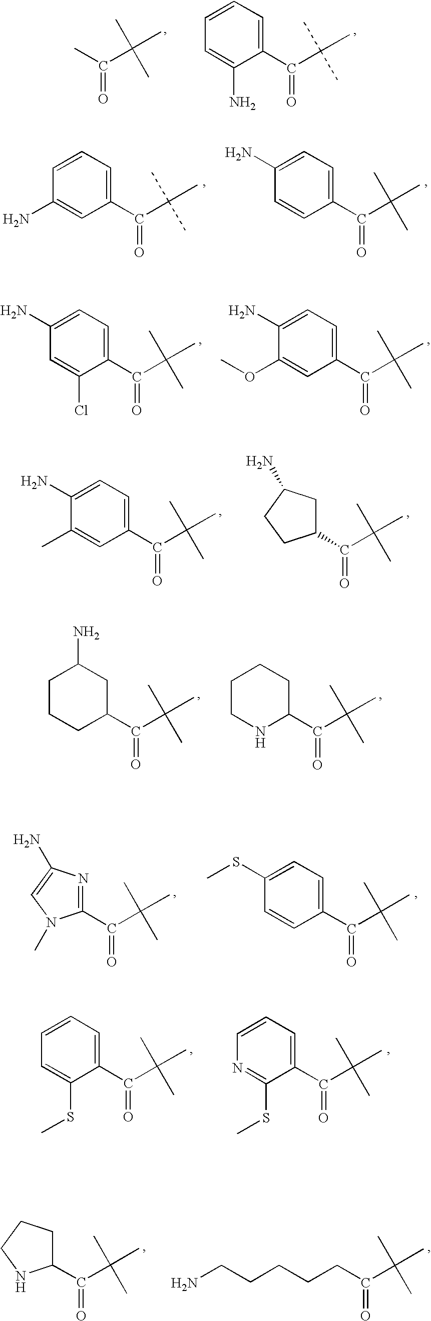

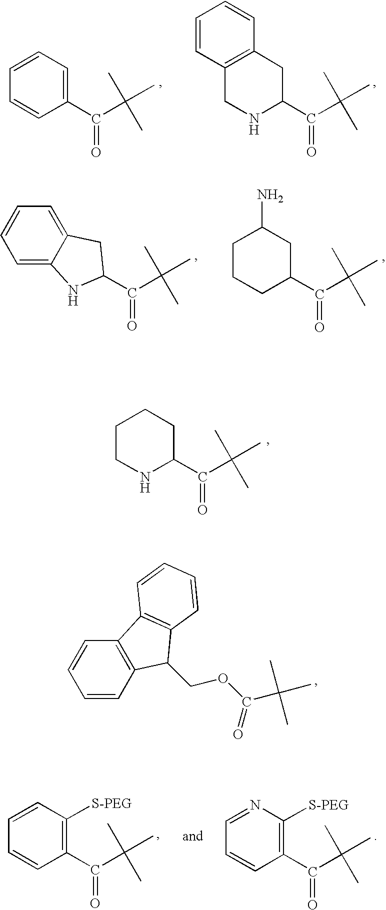

The invention relates to a peptide of Formula (III)

| | |
|---|---|
| Z-YLNLVTRQRY-NH₂ | (SEQ ID NO:3) |
| (III) | |

wherein

Z is selected from

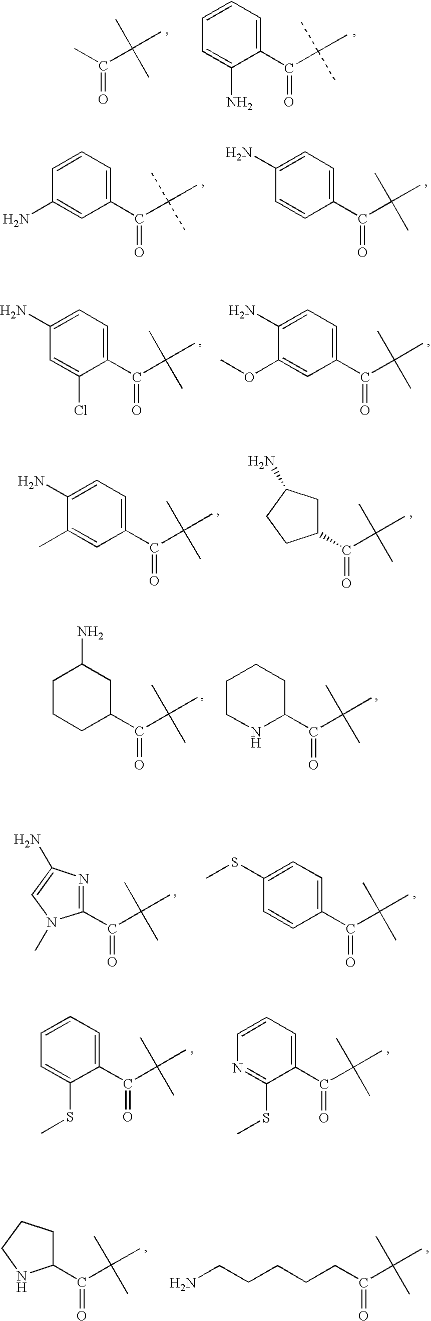

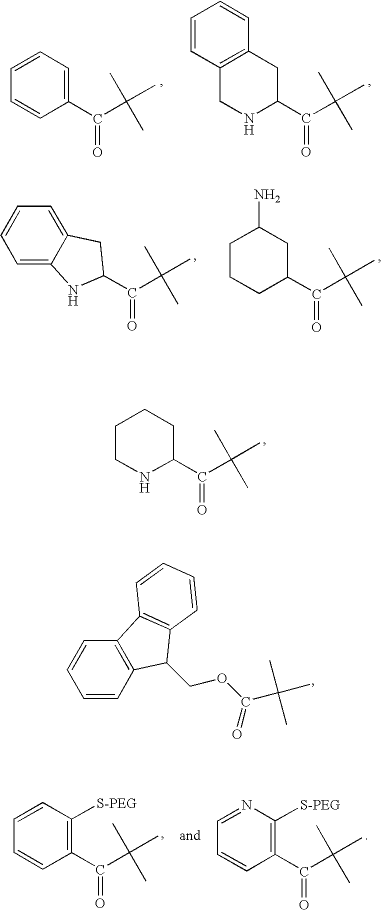

The invention relates to a peptide of Formula (IV)

| | |
|---|---|
| Z-LNLVTRQRY-NH₂ | (SEQ ID NO:4) |
| (IV) | |

wherein

Z is selected from

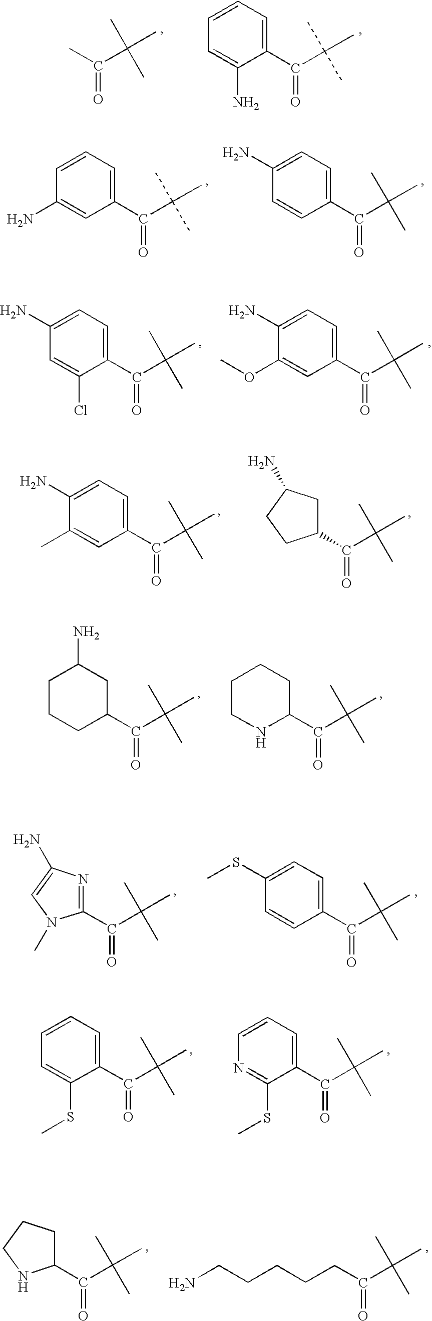

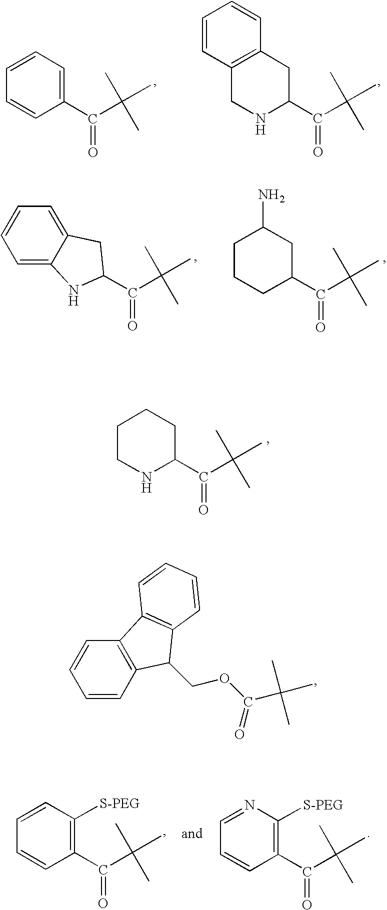

The invention relates to a peptide of Formula (V)

| | |
|---|---|
| Z-NLVTRQRY-NH₂ | (SEQ ID NO:5) |
| (V) | |

wherein

Z is selected from

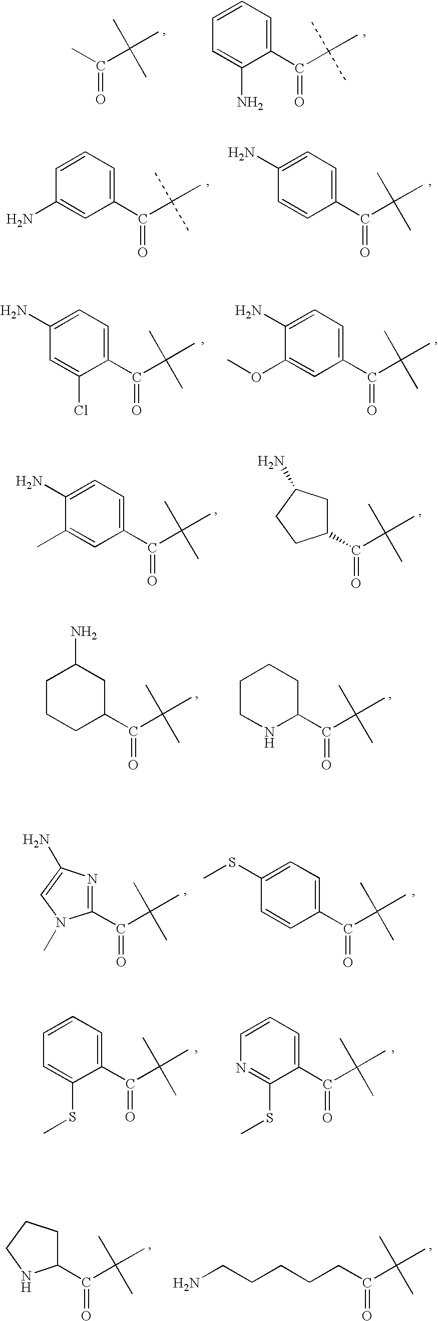

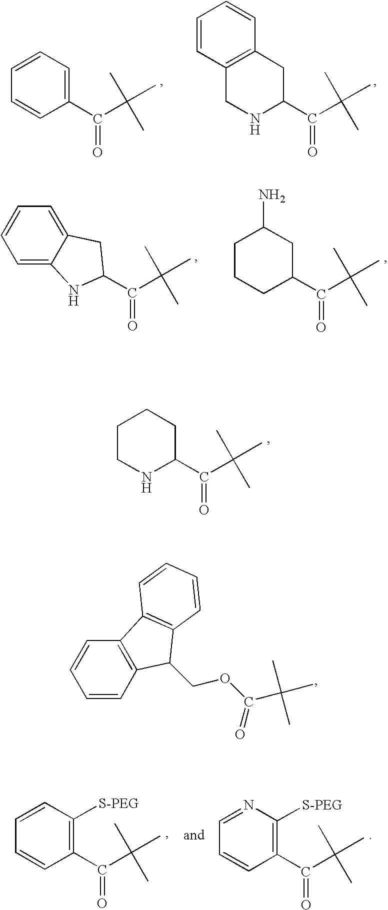

For the peptides of Formula (I), (II), (III), (IV), and (V), the N-terminal modifications are attached via an amide bond to the alpha-amino group of the first amino acid of said peptide.

Certain terms used throughout this specification are defined below, and others will be defined as introduced. The single letter abbreviation for a particular amino acid, its corresponding amino acid, and three letter abbreviation are as follows: A, alanine (Ala); C, cysteine (Cys); D, aspartic acid (Asp); E, glutamic acid (Glu); F, phenylalanine (Phe); G, glycine (Gly); H, histidine (His); I, isoleucine (Ile); K, lysine (Lys); L, leucine (Leu); M, methionine (Met); N, asparagine (Asn); P, proline (Pro); Q, glutamine (Gln); R, arginine (Arg); S, serine (Ser); T, threonine (Thr); V, valine (Val); W, tryptophan (Trp); and Y, tyrosine (Tyr).

The term “polynucleotide encoding a peptide” encompasses a polynucleotide which includes only coding sequence for the peptide, as well as a polynucleotide which includes additional coding and/or non-coding sequence. The present invention further relates to polynucleotides which hybridize to the hereinabove-described sequences if there is at least about 70%, at least about 90%, and at least about 95% identity between the sequences. The present invention relates to polynucleotides encoding peptides which hybridize under stringent conditions to the hereinabove-described polynucleotides. As herein used, the term “stringent conditions” means “stringent hybridization conditions.” Hybridization may occur if there is at least about 90%, about 95%, or about 99% identity between the sequences. The polynucleotides which hybridize to the hereinabove described polynucleotides in one embodiment encode peptides which retain substantially the same biological function or activity as the mature peptide encoded by the cDNAs.

“Functional equivalent” and “substantially the same biological function or activity” each means that degree of biological activity that is within about 30% to about 100% or more of that biological activity demonstrated by the peptide to which it is being compared when the biological activity of each peptide is determined by the same procedure.

“Biological activity,” “activity,” or “biological function,” which are used interchangeably, herein mean an effector function that is directly or indirectly performed by a peptide (whether in its native or denatured conformation), or by any fragments, derivatives, and variants thereof. Biological activities include, for example, binding to polypeptides, binding to other proteins or molecules, activity as a DNA binding protein, as a transcription regulator, ability to bind damaged DNA, etc.

The terms “fragment,” “derivative,” and “variant,” when referring to the peptides of the present invention, means fragments, derivatives, and variants of the peptides which retain substantially the same biological function or activity as such peptides, as described further below.

A fragment is a portion of the peptide which retains substantially similar functional activity, for example, as described in the in vivo models disclosed herein.

A derivative includes all modifications to the peptide which substantially preserve the functions disclosed herein and include additional structure and attendant function (e.g., modified N-terminus peptides or PEGylated peptides), fusion peptides which confer targeting specificity or an additional activity such as toxicity to an intended target, as described further below.

The peptides of the present invention may be recombinant peptides, natural purified peptides, or synthetic peptides.

The fragment, derivative, or variant of the peptides of the present invention may be (i) one in which one or more of the amino acid residues are substituted with a conserved or non-conserved amino acid residue and such substituted amino acid residue may or may not be one encoded by the genetic code, or (ii) one in which one or more of the amino acid residues includes a substituent group, or (iii) one in which the mature peptide is fused with another compound, such as a compound to increase the half-life of the peptide (e.g., polyethylene glycol), or (iv) one in which the additional amino acids are fused to the mature peptide, such as a leader or secretory sequence or a sequence which is employed for purification of the mature peptide, or (v) one in which the peptide sequence is fused with a larger peptide (e.g., human albumin, an antibody or Fc, for increased duration of effect). Such fragments, derivatives, and variants and analogs are deemed to be within the scope of those skilled in the art from the teachings herein.

The derivatives of the present invention may contain conservative amino acid substitutions (defined further below) made at one or more nonessential amino acid residues. A “nonessential” amino acid residue is a residue that can be altered from the wild-type sequence of a protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Fragments, or biologically active portions include peptide fragments suitable for use as a medicament, to generate antibodies, as a research reagent, and the like. Fragments include peptides comprising amino acid sequences sufficiently similar to or derived from the amino acid sequences of a peptide of this invention and exhibiting at least one activity of that peptide, but which include fewer amino acids than the full-length peptides disclosed herein. Typically, biologically active portions comprise a domain or motif with at least one activity of the peptide. A biologically active portion of a peptide can be a peptide which is, for example, five or more amino acids in length. Such biologically active portions can be prepared synthetically or by recombinant techniques and can be evaluated for one or more of the functional activities of a peptide of this invention by means disclosed herein and/or well known in the art.

Moreover, derivatives of the present invention may include peptides that have been fused with another compound, such as a compound to increase the half-life of the peptide and/or to reduce potential immunogenicity of the peptide (e.g., polyethylene glycol, “PEG”). In the case of PEGylation, the fusion of the peptide to PEG may be accomplished by any means known to one skilled in the art. For example, PEGylation may be accomplished by first introducing a cysteine mutation into the peptide to provide a linker upon which to attach the PEG, followed by site-specific derivatization with PEG-maleimide. For example, the cysteine may be added to the C-terminus of the peptides (see, e.g., Tsutsumi, et al., Proc. Natl. Acad. Sci. USA 97(15):8548-53, 2000; Veronese, Biomaterials 22:405-417, 2001; Goodsoon & Katre, Bio/Technology 8:343-346, 1990). Variants of the peptides of this invention include peptides having an amino acid sequence sufficiently similar to the amino acid sequence of the peptides of this invention or a domain thereof. The term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that contain a common structural domain that is at least about 45%, about 75% through 98%, identical are defined herein as sufficiently similar. Variants will be sufficiently similar to the amino acid sequence of the peptides of this invention. Variants include variants of peptides encoded by a polynucleotide that hybridizes to a polynucleotide of this invention or a complement thereof under stringent conditions. Such variants generally retain the functional activity of the peptides of this invention. Libraries of fragments of the polynucleotides may be used to generate a variegated population of fragments for screening and subsequent selection. For example, a library of fragments may be generated by treating a double-stranded PCR fragment of a polynucleotide with a nuclease under conditions wherein nicking occurs only about once per molecule, denaturing the double-stranded DNA, renaturing the DNA to form double-stranded DNA which can include sense/antisense pairs from different nicked products, removing single-stranded portions from reformed duplexes by treatment with S1 nuclease, and ligating the resulting fragment library into an expression vector. By this method, one can derive an expression library that encodes N-terminal and internal fragments of various sizes of the peptide of this invention.

Variants include peptides that differ in amino acid sequence due to mutagenesis. Variants that function as NPY2 receptor agonists may be identified by screening combinatorial libraries of mutants, for example truncation mutants, of the peptides of this invention for NPY2 receptor agonist activity.

In one embodiment, a variegated library of analogs is generated by combinatorial mutagenesis at the nucleic acid level and is encoded by a variegated gene library. A variegated library of variants may be produced by, for example, enzymatically ligating a mixture of synthetic oligonucleotides into gene sequences such that a degenerate set of potential variant amino acid sequences is expressible as individual peptides or alternatively, as a set of larger fusion proteins (e.g., for phage display) containing the set of sequences therein. There are a variety of methods that can be used to produce libraries of potential variants from a degenerate oligonucleotide sequence. Chemical synthesis of a degenerate gene sequence may be performed in an automatic DNA synthesizer, and the synthetic gene then ligated into an appropriate expression vector. Use of a degenerate set of genes allows for the provision, in one mixture, of all of the sequences encoding the desired set of potential variant sequences. Methods for synthesizing degenerate oligonucleotides are known in the art (see, e.g., Narang, Tetrahedron 39:3, 1983; Itakura, et al., Annu. Rev. Biochem. 53:323, 1984; Itakura, et al., Science 198:1056, 1984; Ike, et al., Nucleic Acid Res. 11:477, 1983).

Several techniques are known in the art for screening gene products of combinatorial libraries made by point mutations or truncation and for screening cDNA libraries for gene products having a selected property. Such techniques are adaptable for rapid screening of the gene libraries generated by the combinatorial mutagenesis of R-agonist peptides. The most widely used techniques, which are amenable to high through-put analysis for screening large gene libraries typically include cloning the gene library into replicable expression vectors, transforming appropriate cells with the resulting library of vectors and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify the desired variants.

The invention also provides chimeric or fusion peptides. The targeting sequence is designed to localize the delivery of the peptide to minimize potential side effects. The peptides of this invention may be composed of amino acids joined to each other by peptide bonds or modified peptide bonds (i.e., peptide isosteres), and may contain amino acids other than the 20 gene-encoded amino acids. The peptides may be modified by either natural processes, such as posttranslational processing, or by chemical modification techniques which are well known in the art. Such modifications are well described in basic texts and in more detailed monographs, as well as in a voluminous research literature. Modifications may occur anywhere in a peptide, including the peptide backbone, the amino acid side-chains, and the amino or carboxyl termini. It will be appreciated that the same type of modification may be present in the same or varying degrees at several sites in a given peptide. Also, a given peptide may contain many types of modifications. Peptides may be branched, for example, as a result of ubiquitination, and they may be cyclic, with or without branching. Cyclic, branched, and branched cyclic peptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, PEGylation, proteolytic processing, phosphorylation, prenylation, racemization, selenoylation, sulfation, transfer-RNA mediated addition of amino acids to proteins such as arginylation, and ubiquitination (see, e.g., Proteins, Structure and Molecular Properties, 2nd ed., T. E. Creighton, W.H. Freeman and Company, New York (1993); Posttranslational Covalent Modification of Proteins, B. C. Johnson, ed., Academic Press, New York, pgs. 1-12 (1983); Selfter, et al., Meth. Enzymol 182:626-646, 1990; Rattan, et al., Ann. N.Y. Acad. Sci. 663:48-62, 1992).

The peptides of the present invention include the peptides of Formulae (I), (II), (III), (IV), and (V), as well as those sequences having insubstantial variations in sequence from them. An “insubstantial variation” would include any sequence addition, substitution, or deletion variant that maintains substantially at least one biological function of the peptides of this invention, for example, NPY2 receptor agonist activity, selective NPY2 receptor agonist activity, and/or inhibition of food intake and body weight loss demonstrated herein. These functional equivalents may include peptides which have at least about 90% identity to the peptides of the present invention, at least 95% identity to the peptides of the present invention, and at least 99% identity to the peptides of the present invention, and also include portions of such peptides having substantially the same biological activity. However, any peptide having insubstantial variation in amino acid sequence from the peptides of the present invention that demonstrates functional equivalency as described further herein is included in the description of the present invention.

The present invention also relates to polynucleotides encoding the peptides of this invention, as well as vectors which include these polynucleotides, host cells which are genetically engineered with vectors of the invention, and the production of peptides of the invention by recombinant techniques. Host cells may be genetically engineered (transduced, transformed, or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, or selecting transformants. The culture conditions, such as temperature, pH and the like, are those previously used with the host cell selected for expression, and will be apparent to the ordinarily skilled artisan. The polynucleotide of the present invention may be employed for producing a peptide by recombinant techniques. Thus, for example, the polynucleotide sequence may be included in any one of a variety of expression vehicles, in particular, vectors or plasmids for expressing a peptide. Such vectors include chromosomal, non-chromosomal, and synthetic DNA sequences (e.g., derivatives of SV40); bacterial plasmids; phage DNA; yeast plasmids; vectors derived from combinations of plasmids and phage DNA; viral DNA such as vaccinia, adenovirus, fowl pox virus, and pseudorabies. However, any other vector or plasmid may be used as long as they are replicable and viable in the host.

The appropriate DNA sequence may be inserted into the vector by a variety of procedures. In general, the DNA sequence is inserted into an appropriate restriction endonuclease site by procedures known in the art. Such procedures and others are deemed to be within the scope of those skilled in the art. The DNA sequence in the expression vector is operatively linked to an appropriate expression control sequence(s) (promoter) to direct mRNA synthesis. Representative examples of such promoters include, but are not limited to, LTR or SV40 promoter, the E. coli lac or trp, the phage lambda P_{L} promoter, and other promoters known to control expression of genes in prokaryotic or eukaryotic cells or their viruses. The expression vector may also contain a ribosome binding site for translation initiation and a transcription terminator. The vector may also include appropriate sequences for amplifying expression. In addition, the expression vectors may contain a gene to provide a phenotypic trait for selection of transformed host cells such as dihydrofolate reductase or neomycin resistance for eukaryotic cell culture, or such as tetracycline or ampicillin resistance in E. coli. The vector containing the appropriate DNA sequence as herein above described, as well as an appropriate promoter or control sequence, may be employed to transform an appropriate host to permit the host to express the protein. Representative examples of appropriate hosts, include, but are not limited to, bacterial cells, such as E. coli, Salmonella typhimurium, Streptomyces; fungal cells, such as yeast; insect cells, such as Drosophila S2 and Spodoptera Sf9; animal cells such as CHO, COS, or Bowes melanoma; adenoviruses; plant cells, etc. The selection of an appropriate host is deemed to be within the scope of those skilled in the art from the teachings herein.

The present invention also includes recombinant constructs comprising one or more of the sequences as broadly described above. The constructs comprise a vector, such as a plasmid or viral vector, into which a sequence of the invention has been inserted, in a forward or reverse orientation. In one aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example. Bacterial: pQE70, pQE60, pQE-9, pBS, phagescript, psiX174, pBluescript SK, pBsKS, pNH8a, pNH16a, pNH18a, pNH46a, pTRC99A, pKK223-3, pKK233-3, pDR540, and PRIT5. Eukaryotic: pWLneo, pSV2cat, pOG44, pXT1, pSG, pSVK3, pBPV, pMSG, and PSVL. However, any other plasmid or vector may be used as long as they are replicable and viable in the host. Promoter regions can be selected from any desired gene using CAT (chloramphenicol transferase) vectors or other vectors with selectable markers. Two appropriate vectors are pKK232-8 and pCM7. Particular named bacterial promoters include laci, lacZ, T3, T7, gpt, lambda P_{R}, P_{L}, and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

The present invention also relates to host cells containing the above-described construct. The host cell can be a higher eukaryotic cell such as a mammalian cell or a lower eukaryotic cell such as a yeast cell, or the host cell can be a prokaryotic cell such as a bacterial cell. Introduction of the construct into the host cell can be effected by calcium phosphate transfection, DEAE-Dextran mediated transfection, or electroporation (Davis, et al., Basic Methods in Molecular Biology, 1986). The constructs in host cells can be used in a conventional manner to produce the gene product encoded by the recombinant sequence. Alternatively, the peptides of the invention can be synthetically produced by conventional peptide synthesizers.

Mature proteins may be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, (Cold Spring Harbor, N.Y., 1989), the disclosure of which is hereby incorporated by reference.

Transcription of a DNA encoding the peptides of the present invention by higher eukaryotes is increased by inserting an enhancer sequence into the vector. Enhancers are cis-acting elements of DNA, usually from about 10 to about 300 bp, that act on a promoter to increase its transcription. Examples include the SV40 enhancer on the late side of the replication origin (bp 100 to 270), a cytomegalovirus early promoter enhancer, a polyoma enhancer on the late side of the replication origin, and adenovirus enhancers. Generally, recombinant expression vectors will include origins of replication and selectable markers permitting transformation of the host cell (e.g., the ampicillin resistance gene of E. coli or S. cerevisiae TRP1 gene), and a promoter derived from a highly-expressed gene to direct transcription of a downstream structural sequence. Such promoters can be derived from operons encoding glycolytic enzymes such as 3-phosphoglycerate kinase (PGK), α factor, acid phosphatase, or heat shock proteins, among others. The heterologous structural sequence is assembled in appropriate phase with translation, initiation and termination sequences, and optionally a leader sequence capable of directing secretion of translated protein into the periplasmic space or extracellular medium. Optionally, the heterologous sequence may encode a fusion protein including an N-terminal identification peptide imparting desired characteristics (e.g., stabilization or simplified purification of expressed recombinant product).

Useful expression vectors for bacterial use may be constructed by inserting a structural DNA sequence encoding a desired protein together with suitable translation, initiation, and termination signals in operable reading phase with a functional promoter. The vector may comprise one or more phenotypic selectable markers and an origin of replication to ensure maintenance of the vector and to, if desirable, provide amplification within the host. Suitable prokaryotic hosts for transformation include, for example, E. coli, Bacillus subtilis, Salmonella typhimurium, and various species within the genera Pseudomonas, Streptomyces, and Staphylococcus, although others may also be employed as a matter of choice. Useful expression vectors for bacterial use may comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega, Madison, Wis., USA). These pBR322 “backbone” sections may be combined with an appropriate promoter and the structural sequence to be expressed.

After transformation of a suitable host strain and growth of the host strain to an appropriate cell density, the selected promoter is derepressed by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents.

Various mammalian cell culture systems may also be employed to express recombinant protein. Examples of mammalian expression systems include the COS-7 lines of monkey kidney fibroblasts described by Gluzman, (Cell 23:175, 1981), and other cell lines capable of expressing a compatible vector, for example, the C127, 3T3, CHO, HeLa, and BHK cell lines. Mammalian expression vectors may comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required non-transcribed genetic elements.

The peptides of the present invention may be recovered and purified from recombinant cell cultures by methods used heretofore, including ammonium sulfate or ethanol precipitation, acid extraction, anion or cation exchange chromatography, phosphocellulose chromatography, hydrophobic interaction chromatography, affinity chromatography, hydroxyapatite chromatography, and lectin chromatography. Protein refolding steps can be used, as necessary, in completing configuration of the mature protein. Finally, high performance liquid chromatography (HPLC) may be employed for final purification steps.

The peptides of this invention may be a product of chemical synthetic procedures, or produced by recombinant techniques from a prokaryotic or eukaryotic host (e.g., bacterial, yeast, higher plant, insect, and mammalian cells). Depending upon the host employed in a recombinant production procedure, the peptides of this invention may be glycosylated with mammalian or other eukaryotic carbohydrates, or may be non-glycosylated. Peptides of this invention may also include an initial methionine amino acid residue.

The peptides of this invention may be conveniently isolated by methods that are well known in the art. Purity of the preparations may also be assessed by any means known in the art, such as SDS-polyacrylamide gel electrophoresis and mass spectroscopy and liquid chromatography.

Polynucleotide sequences encoding a peptide of this invention may be synthesized, in whole or in part, using chemical methods well known in the art (see, e.g., Caruthers, et al., Nucl. Acids Res. Symp. Ser. 215-223, 1980; Horn, et al., Nucl. Acids Res. Symp. Ser. 225-232, 1980). The polynucleotide that encodes the peptide may then be cloned into an expression vector to express the peptide.

As will be understood by those of skill in the art, it may be advantageous to produce the peptide-encoding nucleotide sequences possessing non-naturally occurring codons. For example, codons preferred by a particular prokaryotic or eukaryotic host can be selected to increase the rate of peptide expression or to produce an RNA transcript having desirable properties, such as a half-life which is longer than that of a transcript generated from the naturally occurring sequence.

Nucleotide sequences may be engineered using methods generally known in the art to alter the peptide-encoding sequences for a variety of reasons, including but not limited to, alterations which modify the closing, processing, and/or expression of the peptide or mRNA product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, introduce mutations, and so forth.

Also provided are related peptides within the understanding of those with skill in the art, such as chemical mimetics, organomimetics, or peptidomimetics. As used herein, the terms “mimetic,” “peptide mimetic,” “peptidomimetic,” “organomimetic,” and “chemical mimetic” are intended to encompass peptide derivatives, peptide analogs, and chemical compounds having an arrangement of atoms in a three-dimensional orientation that is equivalent to that of a peptide of the present invention. It will be understood that the phrase “equivalent to” as used herein is intended to encompass peptides having substitution(s) of certain atoms, or chemical moieties in said peptide, having bond lengths, bond angles, and arrangements in the mimetic peptide that produce the same or sufficiently similar arrangement or orientation of said atoms and moieties to have the biological function of the peptides of the invention. In peptide mimetics, the three-dimensional arrangement of the chemical constituents may be structurally and/or functionally equivalent to the three-dimensional arrangement of the peptide backbone and component amino acid sidechains in the peptide, resulting in such peptido-, organs, and chemical mimetics of the peptides of the invention having substantial biological activity. These terms are used according to the understanding in the art, as illustrated, for example, by Fauchere, (Adv. Drug Res. 15:29, 1986); Veber & Freidinger, (TINS p. 392, 1985); and Evans, et al., (J. Med. Chem. 30:1229, 1987), incorporated herein by reference.

It is understood that a pharmacophore exists for the biological activity of each peptide of the invention. A pharmacophore is understood in the art as comprising an idealized, three-dimensional definition of the structural requirements for biological activity. Peptido-, organo-, and chemical mimetics may be designed to fit each pharmacophore with current computer modeling software (computer aided drug design). Said mimetics may be produced by structure-function analysis, based on the positional information from the substituent atoms in the peptides of the invention.

Peptides as provided by the invention may be advantageously synthesized by any of the chemical synthesis techniques known in the art, particularly solid-phase synthesis techniques, for example, using commercially-available automated peptide synthesizers. The mimetics of the present invention may be synthesized by solid phase or solution phase methods conventionally used for the synthesis of peptides (see, e.g., Merrifield, J. Amer. Chem. Soc. 85:2149-54, 1963; Carpino, Acc. Chem. Res. 6:191-98, 1973; Birr, Aspects of the Merrifield Peptide Synthesis, Springer-Verlag: Heidelberg, 1978; The Peptides: Analysis, Synthesis, Biology, Vols. 1, 2, 3, and 5, (Gross & Meinhofer, eds.), Academic Press: New York, 1979; Stewart, et al., Solid Phase Peptide Synthesis, 2nd. ed., Pierce Chem. Co.: Rockford, Ill., 1984; Kent, Ann. Rev. Biochem. 57:957-89, 1988; and Gregg, et al., Int. J. Peptide Protein Res. 55:161-214, 1990, which are incorporated herein by reference in their entirety.)

Peptides of the present invention may be prepared by solid phase methodology. Briefly, an N-protected C-terminal amino acid residue is linked to an insoluble support such as divinylbenzene cross-linked polystyrene, polyacrylamide resin, Kieselguhr/polyamide (pepsyn K), controlled pore glass, cellulose, polypropylene membranes, acrylic acid-coated polyethylene rods, or the like. Cycles of deprotection, neutralization, and coupling of successive protected amino acid derivatives are used to link the amino acids from the C-terminus according to the amino acid sequence. For some synthetic peptides, an FMOC strategy using an acid-sensitive resin may be used. Solid supports in this regard may be divinylbenzene cross-linked polystyrene resins, which are commercially available in a variety of functionalized forms, including chloromethyl resin, hydroxymethyl resin, paraacetamidomethyl resin, benzhydrylamine (BHA) resin, 4-methylbenzhydrylamine (MBHA) resin, oxime resins, 4-alkoxybenzyl alcohol resin (Wang resin), 4-(2′,4′-dimethoxyphenylaminomethyl)-phenoxymethyl resin, 2,4-dimethoxybenzhydryl-amine resin, and 4-(2′,4′-dimethoxyphenyl-FMOC-amino-methyl)-phenoxyacetamidonorleucyl-MBHA resin (Rink amide MBHA resin). In addition, acid-sensitive resins also provide C-terminal acids, if desired. A protecting group for alpha amino acids is base-labile fluorenylmethoxy-carbonyl (FMOC).

Suitable protecting groups for the side chain functionalities of amino acids chemically compatible with BOC (t-butyloxycarbonyl) and FMOC groups are well known in the art. When using FMOC chemistry, the following protected amino acid derivatives may be utilized: FMOC-Cys(Trit), FMOC-Ser(But), FMOC-Asn(Trit), FMOC-Leu, FMOC-Thr(Trit), FMOC-Val, FMOC-Gly, FMOC-Lys(Boc), FMOC-Gln(Trit), FMOC-Glu(OBut), FMOC-His(Trit), FMOC-Tyr(But), FMOC-Arg(PMC (2,2,5,7,8-pentamethylchroman-6-sulfonyl)), FMOC-Arg(BOC)₂, FMOC-Pro, and FMOC, Trp(BOC). The amino acid residues may be coupled by using a variety of coupling agents and chemistries known in the art, such as direct coupling with DIC (diisopropyl-carbodiimide), DCC (dicyclohexylcarbodiimide), BOP (benzotriazolyl-N-oxytrisdimethylaminophosphonium hexafluorophosphate), PyBOP (benzotriazole-1-yl-oxy-tris-pyrrolidinophosphonium hexafluorophosphate), PyBrOP (bromo-tris-pyrrolidinophosphonium hexafluorophosphate); via performed symmetrical anhydrides; via active esters such as pentafluorophenyl esters; or via performed HOBt (1-hydroxybenzotriazole) active esters or by using FMOC-amino acid fluoride and chlorides or by using FMOC-amino acid-N-carboxy anhydrides. Activation with HBTU (2-(1H-benzotriazole-1-yl),1,1,3,3-tetramethyluronium hexafluorophosphate) or HATU (2-(1H-7-aza-benzotriazole-1-yl),1,1,3,3-tetramethyluronium hexafluoro-phosphate) in the presence of HOBt or HOAt (7-azahydroxybenztriazole) is preferred.

The solid phase method may be carried out manually, or by automated synthesis on a commercially available peptide synthesizer (e.g., Applied Biosystems 431A or the like; Applied Biosystems, Foster City, Calif.). In a typical synthesis, the first (C-terminal) amino acid is loaded on the chlorotrityl resin. Successive deprotection (with 20% piperidine/NMP (N-methylpyrrolidone)) and coupling cycles according to ABI FastMoc protocols (Applied Biosystems) may be used to generate the peptide sequence. Double and triple coupling, with capping by acetic anhydride, may also be used.

The synthetic mimetic peptide may be cleaved from the resin and deprotected by treatment with TFA (trifluoroacetic acid) containing appropriate scavengers. Many such cleavage reagents, such as Reagent K (0.75 g crystalline phenol, 0.25 mL ethanedithiol, 0.5 mL thioanisole, 0.5 mL deionized water, 10 mL TFA) and others, may be used. The peptide is separated from the resin by filtration and isolated by ether precipitation. Further purification may be achieved by conventional methods, such as gel filtration and reverse phase HPLC (high performance liquid chromatography). Synthetic mimetics according to the present invention may be in the form of pharmaceutically acceptable salts, especially base-addition salts including salts of organic bases and inorganic bases. The base-addition salts of the acidic amino acid residues are prepared by treatment of the peptide with the appropriate base or inorganic base, according to procedures well known to those skilled in the art, or the desired salt may be obtained directly by lyophilization of the appropriate base.

Generally, those skilled in the art will recognize that peptides as described herein may be modified by a variety of chemical techniques to produce peptides having essentially the same activity as the unmodified peptide, and optionally having other desirable properties. For example, carboxylic acid groups of the peptide may be provided in the form of a salt of a pharmaceutically-acceptable cation. Amino groups within the peptide may be in the form of a pharmaceutically-acceptable acid addition salt, such as the HCl, HBr, acetic, benzoic, toluene sulfonic, maleic, tartaric, and other organic salts, or may be converted to an amide. Thiols may be protected with any one of a number of well-recognized protecting groups, such as acetamide groups. Those skilled in the art will also recognize methods for introducing cyclic structures into the peptides of this invention so that the native binding configuration will be more nearly approximated. For example, a carboxyl terminal or amino terminal cysteine residue may be added to the peptide, so that when oxidized the peptide will contain a disulfide bond, thereby generating a cyclic peptide. Other peptide cyclizing methods include the formation of thioethers and carboxyl- and amino-terminal amides and esters.

Specifically, a variety of techniques are available for constructing peptide derivatives and analogs with the same or similar desired biological activity as the corresponding peptide but with more favorable activity than the peptide with respect to solubility, stability, and susceptibility to hydrolysis and proteolysis. Such derivatives and analogs include peptides modified at the N-terminal amino group, as exemplified by, but not limited to, the peptides of Formulae (I), (II), (III), (IV), and (V), the C-terminal amide group, and/or changing one or more of the amido linkages in the peptide to a non-amido linkage. It will be understood that two or more such modifications may be coupled in one peptide mimetic structure (e.g., modification at the C-terminal amide group and inclusion of a —CH₂— carbamate linkage between two amino acids in the peptide).

Peptide mimetics as understood in the art and provided by the invention are structurally similar to the peptides of the invention, but have one or more peptide linkages optionally replaced by a linkage, for example, —CH₂NH—, —CH₂S—, —CH₂CH₂—, —CH═CH— (in both cis and trans conformers), —COCH₂—, —CH(OH)CH₂—, and —CH₂SO—, by methods known in the art and further described in the following references: Spatola, Chemistry and Biochemistry of Amino Acids, Peptides, and Proteins, (Weinstein, ed.), Marcel Dekker: New York, p. 267, 1983; Spatola, Peptide Backbone Modifications 1:3, 1983; Morley, Trends Pharm. Sci. pp. 463-468, 1980; Hudson, et al., Int. J. Pept. Prot. Res. 14:177-185, 1979; Spatola, et al., Life Sci. 38:1243-1249, 1986; Hann, J. Chem. Soc. Perkin Trans. 1307-314, 1982; Almquist, et al., J. Med. Chem. 23:1392-1398, 1980; Jennings-White, et al., Tetrahedron Lett. 23:2533, 1982; Szelke, et al., EP045665A; Holladay, et al., Tetrahedron Lett. 24:4401-4404, 1983; and Hruby, Life Sci. 31:189-199, 1982; each of which is incorporated herein by reference. Such peptide mimetics may have significant advantages over peptide embodiments, including, for example, more economical to produce, having greater chemical stability or enhanced pharmacological properties (such as half-life, absorption, potency, efficacy, etc.), reduced antigenicity, and other properties.

Mimetic analogs of the peptides of the invention may also be obtained using the principles of conventional or rational drug design (see, e.g., Andrews, et al., Proc. Alfred Benzon Symp. 28:145-165, 1990; McPherson, Eur. J. Biochem. 189:1-24, 1990; Hol, et al., in Molecular Recognition: Chemical and Biochemical Problems, (Roberts, ed.); Royal Society of Chemistry, pp. 84-93, 1989a; Hol, Arzneim-Forsch. 39:1016-1018, 1989b; Hol, Agnew Chem. Int. Ed. Engl. 25:767-778, 1986; the disclosures of which are herein incorporated by reference).

In accordance with the methods of conventional drug design, the desired mimetic molecules may be obtained by randomly testing molecules whose structures have an attribute in common with the structure of a “native” peptide. The quantitative contribution that results from a change in a particular group of a binding molecule may be determined by measuring the biological activity of the putative mimetic in comparison with the activity of the peptide. In one embodiment of rational drug design, the mimetic is designed to share an attribute of the most stable three-dimensional conformation of the peptide. Thus, for example, the mimetic may be designed to possess chemical groups that are oriented in a way sufficient to cause ionic, hydrophobic, or van der Waals interactions that are similar to those exhibited by the peptides of the invention, as disclosed herein.

One method for performing rational mimetic design employs a computer system capable of forming a representation of the three-dimensional structure of the peptide, such as those exemplified by Hol, 1989a; Hol, 1989b; and Hol, 1986. Molecular structures of the peptido-, organo-, and chemical mimetics of the peptides of the invention may be produced using computer-assisted design programs commercially available in the art. Examples of such programs include SYBYL 6.5®, HQSAR™, and ALCHEMY 2000™ (Tripos); GALAXY™ and AM2000™ (AM Technologies, Inc., San Antonio, Tex.); CATALYST™ and CERIUS™ (Molecular Simulations, Inc., San Diego, Calif.); CACHE PRODUCTST™, TSAR™, AMBER™, and CHEM-X™ (Oxford Molecular Products, Oxford, Calif.) and CHEMBUILDER3D™ (Interactive Simulations, Inc., San Diego, Calif.).

The peptido-, organo-, and chemical mimetics produced using the peptides disclosed herein using, for example, art-recognized molecular modeling programs may be produced using conventional chemical synthetic techniques, for example, designed to accommodate high throughput screening, including combinatorial chemistry methods. Combinatorial methods useful in the production of the peptido-, organo-, and chemical mimetics of the invention include phage display arrays, solid-phase synthesis, and combinatorial chemistry arrays, as provided, for example, by SIDDCO (Tuscon, Ariz.); Tripos, Inc.; Calbiochem/Novabiochem (San Diego, Calif.); Symyx Technologies, Inc. (Santa Clara, Calif.); Medichem Research, Inc. (Lemont, Ill.); Pharm-Eco Laboratories, Inc. (Bethlehem, Pa.); or N.V. Organon (Oss, Netherlands). Combinatorial chemistry production of the peptido-, organs, and chemical mimetics of the invention may be produced according to methods known in the art, including, but not limited to, techniques disclosed in Terrett, (Combinatorial Chemistry, Oxford University Press, London, 1998); Gallop, et al., J. Med. Chem. 37:1233-51, 1994; Gordon, et al., J. Med. Chem. 37:1385-1401, 1994; Look, et al., Bioorg. Med. Chem. Lett. 6:707-12, 1996; Ruhland, et al., J. Amer. Chem. Soc. 118: 253-4, 1996; Gordon, et al., Acc. Chem. Res. 29:144-54, 1996; Thompson & Ellman, Chem. Rev. 96:555-600, 1996; Fruchtel & Jung, Angew. Chem. Int. Ed. Engl. 35:17-42, 1996; Pavia, “The Chemical Generation of Molecular Diversity”, Network Science Center, www.netsci.org, 1995; Adnan, et al., “Solid Support Combinatorial Chemistry in Lead Discovery and SAR Optimization,” Id., 1995; Davies and Briant, “Combinatorial Chemistry Library Design using Pharmacophore Diversity,” Id., 1995; Pavia, “Chemically Generated Screening Libraries: Present and Future,” Id., 1996; and U.S. Pat. Nos. 5,880,972; 5,463,564; 5,331,573; and 5,573,905.

The newly synthesized peptides may be substantially purified by preparative high performance liquid chromatography (see, e.g., Creighton, Proteins: Structures And Molecular Principles, WH Freeman and Co., New York, N.Y., 1983). The composition of a synthetic peptide of the present invention may be confirmed by amino acid analysis or sequencing by, for example, the Edman degradation procedure (Creighton, supra). Additionally, any portion of the amino acid sequence of the peptide may be altered during direct synthesis and/or combined using chemical methods with sequences from other proteins to produce a variant peptide or a fusion peptide.

Also included in this invention are antibodies and antibody fragments that selectively bind the peptides of this invention. Any type of antibody known in the art may be generated using methods well known in the art. For example, an antibody may be generated to bind specifically to an epitope of a peptide of this invention. “Antibody” as used herein includes intact immunoglobulin molecules, as well as fragments thereof, such as Fab, F(ab′)₂, and Fv, which are capable of binding an epitope of a peptide of this invention. Typically, at least 6, 8, 10, or 12 contiguous amino acids are required to form an epitope. However, epitopes which involve non-contiguous amino acids may require more amino acids, for example, at least 15, 25, or 50 amino acids.

An antibody which specifically binds to an epitope of a peptide of this invention may be used therapeutically, as well as in immunochemical assays, such as Western blots, ELISAs, radioimmunoassays, immunohistochemical assays, immunoprecipitations, or other immunochemical assays known in the art. Various immunoassays may be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a peptide of this invention provides a detection signal higher than a detection signal provided with other proteins when used in an immunochemical assay. Antibodies which specifically bind to a peptide of this invention do not detect other proteins in immunochemical assays and can immunoprecipitate a peptide of this invention from solution.

Peptides of this invention may be used to immunize a mammal, such as a mouse, rat, rabbit, guinea pig, monkey, or human, to produce polyclonal antibodies. If desired, a peptide of this invention may be conjugated to a carrier protein, such as bovine serum albumin, thyroglobulin, and keyhole limpet hemocyanin. Depending on the host species, various adjuvants may be used to increase the immunological response. Such adjuvants include, but are not limited to, Freund's adjuvant, mineral gels (e.g., aluminum hydroxide), and surface active substances (e.g., lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a peptide of this invention may be prepared using any technique which provides for the production of antibody molecules by continuous cell lines in culture. These techniques include, but are not limited to, the hybridoma technique, the human B cell hybridoma technique, and the EBV hybridoma technique (Kohler, et al., Nature 256:495-97, 1985; Kozbor, et al., J. Immunol. Methods 81:3142, 1985; Cote, et al., Proc. Natl. Acad. Sci. 80:2026-30, 1983; Cole, et al., Mol. Cell Biol. 62:109-20, 1984).

In addition, techniques developed for the production of “chimeric antibodies,” the splicing of mouse antibody genes to human antibody genes to obtain a molecule with appropriate antigen specificity and biological activity, may be used (Morrison, et al., Proc. Natl. Acad. Sci. 81:6851-55, 1984; Neuberger, et al., Nature 312:604-08, 1984; Takeda, et al., Nature 314:452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences may be minimized by replacing residues which differ from those in the human sequences by site directed mutagenesis of individual residues or by grating of entire complementarity determining regions. Alternatively, humanized antibodies may be produced using recombinant methods (see, e.g., GB2188638B). Antibodies which specifically bind to a peptide of this invention may contain antigen binding sites which are either partially or fully humanized, as disclosed in U.S. Pat. No. 5,565,332.

Alternatively, techniques described for the production of single chain antibodies may be adapted using methods known in the art to produce single chain antibodies which specifically bind to a peptide of this invention. Antibodies with related specificity, but of distinct idiotypic composition, can be generated by chain shuffling from random combinatorial immunoglobin libraries (Burton, Proc. Natl. Acad. Sci. 88:11120-23, 1991).

Single-chain antibodies also may be constructed using a DNA amplification method, such as PCR, using hybridoma cDNA as a template (Thirion, et al., Eur. J. Cancer Prev. 5:507-11, 1996). Single-chain antibodies can be mono- or bispecific, and can be bivalent or tetravalent. Construction of tetravalent, bispecific single-chain antibodies is taught, for example, in Coloma & Morrison (Nat. Biotechnol. 15:159-63, 1997). Construction of bivalent, bispecific single-chain antibodies is taught in Mallender & Voss (J. Biol. Chem. 269:199-206, 1994).

A nucleotide sequence encoding a single-chain antibody may be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar, et al., Int. J. Cancer 61:497-501, 1995; Nicholls, et al., J. Immunol. Meth. 165:81-91, 1993).

Antibodies which specifically bind to a peptide of this invention may also be produced by inducing in vivo production in the lymphocyte population or by screening immunoglobulin libraries or panels of highly specific binding reagents as disclosed in the literature (Orlandi, et al., Proc. Natl. Acad. Sci. 86:38333-37, 1989; Winter, et al., Nature 349:293-99, 1991).

Other types of antibodies may be constructed and used therapeutically in methods of the invention. For example, chimeric antibodies may be constructed as disclosed in WO 93/03151. Binding proteins which are derived from immunoglobulins and which are multivalent and multispecific, such as the “diabodies” also can be prepared (see, e.g., WO 94/13804,).

Human antibodies with the ability to bind to the peptides of this invention may also be identified from the MorphoSys HuCAL® library as follows. A peptide of this invention may be coated on a microtiter plate and incubated with the MorphoSys HuCAL® Fab phage library. Those phage-linked Fabs not binding to the peptide of this invention can be washed away from the plate, leaving only phage which tightly bind to the peptide of this invention. The bound phage can be eluted, for example, by a change in pH or by elution with E. coli and amplified by infection of E. coli hosts. This panning process can be repeated once or twice to enrich for a population of antibodies that tightly bind to the peptide of this invention. The Fabs from the enriched pool are then expressed, purified, and screened in an ELISA assay.

Antibodies according to the invention may be purified by methods well known in the art. For example, antibodies may be affinity purified by passage over a column to which a peptide of this invention is bound. The bound antibodies can then be eluted from the column using a buffer with a high salt concentration.

### Methods of Use

As used herein, various terms are defined below.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “subject” as used herein includes mammals (e.g., humans and animals).

The term “treatment” includes any process, action, application, therapy, or the like, wherein a subject, including a human being, is provided medical aid with the object of improving the subject's condition, directly or indirectly, or slowing the progression of a condition or disorder in the subject.

The term “combination therapy or co-therapy” means the administration of two or more therapeutic agents to treat, for example, an obese condition and/or disorder. Such administration encompasses co-administration of two or more therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner.

The phrase “therapeutically effective” means the amount of each agent administered that will achieve the goal of improvement in an obese condition or disorder severity, while avoiding or minimizing adverse side effects associated with the given therapeutic treatment.

The term “pharmaceutically acceptable” means that the subject item is appropriate for use in a pharmaceutical product.

The peptides of Formulae (I), (II), (III), (IV), and (V) are expected to be valuable as therapeutic agents. Accordingly, an embodiment of this invention includes a method of treating the various conditions in a patient (including mammals) which comprises administering to said patient a composition containing an amount of the peptide of Formulae (I), (II), (III), (IV), or (V) that is effective in treating the target condition.

The peptides of the present invention interact with the NPY2 receptor and may be used in the treatment or prevention of diseases and/or behaviors that involve the NPY2 receptor.

For example, an object of this invention is to provide methods for treating obesity and inducing weight loss in an individual by administration of a peptide of the invention. The method of the invention comprises administering to an individual a therapeutically effective amount of at least one peptide of the invention which is sufficient to induce weight loss. The invention further comprises a method of preventing weight gain in an individual by administering an amount of at least one peptide of the invention which is sufficient to prevent weight gain.

The present invention also relates to the use of the peptides of this invention for the treatment of obesity-related diseases including associated dyslipidemia and other obesity- and overweight-related complications such as, for example, cholesterol gallstones, gallbladder disease, gout, cancer (e.g., colon, rectum, prostate, breast, ovary, endometrium, cervix, gallbladder, and bile duct), menstrual abnormalities, infertility, polycystic ovaries, osteoarthritis, and sleep apnea, as well as for a number of other pharmaceutical uses associated therewith, such as the regulation of appetite and food intake, dyslipidemia, hypertriglyceridemia, Syndrome X, type 2 diabetes (non-insulin-dependent diabetes), atherosclerotic diseases such as heart failure, hyperlipidemia, hypercholesteremia, low HDL levels, hypertension, cardiovascular disease (including atherosclerosis, coronary heart disease, coronary artery disease, and hypertension), cerebrovascular disease such as stroke, and peripheral vessel disease. The peptides of this invention may also be useful for treating physiological disorders related to, for example, regulation of insulin sensitivity, inflammatory response, plasma triglycerides, HDL, LDL and cholesterol levels and the like.

The peptides of the present invention may be administered alone or in combination with one or more additional therapeutic agents. Combination therapy includes administration of a single pharmaceutical dosage formulation which contains a peptide of the present invention and one or more additional therapeutic agents, as well as administration of a peptide of the present invention and each additional therapeutic agents in its own separate pharmaceutical dosage formulation. For example, a peptide of the present invention and a therapeutic agent may be administered to the patient together in a single oral dosage composition such as a tablet or capsule, or each agent may be administered in separate oral dosage formulations.

Where separate dosage formulations are used, a peptide of the present invention and one or more additional therapeutic agents may be administered at essentially the same time (e.g., concurrently) or at separately staggered times (e.g., sequentially).

Peptides of the invention may also be used in combination with anti-obesity drugs. For example, anti-obesity drugs include β-3 agonists such as CL 316,243; cannabinoid (e.g., CB-1) antagonists; appetite suppressants, such as, for example, sibutramine (Meridia); and lipase inhibitors, such as, for example, orlistat (Xenical). The peptides of the present invention may also be administered in combination with a drug compound that modulates digestion and/or metabolism such as drugs that modulate thermogenesis, lipolysis, gut motility, fat absorption, and satiety.

In addition, the peptides of the present invention may be administered in combination with one or more of the following agents for the treatment of diabetes or diabetes-related disorders including PPAR ligands (agonists, antagonists), insulin secretagogues, for example, sulfonylurea drugs and non-sulfonylurea secretagogues, α-glucosidase inhibitors, insulin sensitizers, hepatic glucose output lowering compounds, and insulin and insulin derivatives. Such therapies may be administered prior to, concurrently with, or following administration of the peptides of the invention. Insulin and insulin derivatives include both long and short acting forms and formulations of insulin. PPAR ligands may include agonists and/or antagonists of any of the PPAR receptors or combinations thereof. For example, PPAR ligands may include ligands of PPAR-α, PPAR-γ, PPAR-δ or any combination of two or three of the receptors of PPAR. PPAR ligands include, for example, rosiglitazone, troglitazone, and pioglitazone. Sulfonylurea drugs include, for example, glyburide, glimepiride, chlorpropamide, tolbutamide, and glipizide. α-glucosidase inhibitors that may be useful in treating diabetes when administered with a peptide of the invention include acarbose, miglitol, and voglibose. Insulin sensitizers that may be useful in treating diabetes include PPAR-γ agonists such as the glitazones (e.g., troglitazone, pioglitazone, englitazone, MCC-555, rosiglitazone, and the like) and other thiazolidinedione and non-thiazolidinedione compounds; biguanides such as metformin and phenformin; protein tyrosine phosphatase-1B (PTP-1B) inhibitors; dipeptidyl peptidase IV (DPP-IV) inhibitors, and 11beta-HSD inhibitors. Hepatic glucose output lowering compounds that may be useful in treating diabetes when administered with a peptide of the invention include glucagon anatgonists and metformin, such as Glucophage and Glucophage XR. Insulin secretagogues that may be useful in treating diabetes when administered with a peptide of the invention include sulfonylurea and non-sulfonylurea drugs: GLP-1, GIP, PACAP, secretin, and derivatives thereof; nateglinide, meglitinide, repaglinide, glibenclamide, glimepiride, chlorpropamide, glipizide. GLP-1 includes derivatives of GLP-1 with longer half-lives than native GLP-1, such as, for example, fatty-acid derivatized GLP-1 and exendin.

Peptides of the invention may also be used in methods of the invention in combination with drugs commonly used to treat lipid disorders in patients. Such drugs include, but are not limited to, HMG-CoA reductase inhibitors, nicotinic acid, fatty acid lowering compounds (e.g., acipimox); lipid lowering drugs (e.g., stanol esters, sterol glycosides such as tiqueside, and azetidinones such as ezetimibe), ACAT inhibitors (such as avasimibe), bile acid sequestrants, bile acid reuptake inhibitors, microsomal triglyceride transport inhibitors, and fibric acid derivatives. HMG-CoA reductase inhibitors include, for example, lovastatin, simvastatin, pravastatin, fluvastatin, atorvastatin, rivastatin, itavastatin, cerivastatin, and ZD-04522. Fibric acid derivatives include, for example, clofibrate, fenofibrate, bezafibrate, ciprofibrate, beclofibrate, etofibrate, and gemfibrozil. Sequestrants include, for example, cholestyramine, colestipol, and dialkylaminoalkyl derivatives of a cross-linked dextran.

Peptides of the invention may also be used in combination with anti-hypertensive drugs, such as, for example, β-blockers and ACE inhibitors. Examples of additional anti-hypertensive agents for use in combination with the peptides of the present invention include calcium channel blockers (L-type and T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan, neutral endopeptidase (NEP) inhibitors, vasopepsidase inhibitors (dual NEP-ACE inhibitors) (e.g., omapatrilat and gemopatrilat), and nitrates.

### Pharmaceutical Compositions

Based on well known assays used to determine the efficacy for treatment of conditions identified above in mammals, and by comparison of these results with the results of known medicaments that are used to treat these conditions, the effective dosage of the peptides of this invention can readily be determined for treatment of each desired indication. The amount of the active ingredient to be administered in the treatment of one of these conditions can vary widely according to such considerations as the particular peptide and dosage unit employed, the mode of administration, the period of treatment, the age and sex of the patient treated, and the nature and extent of the condition treated.

The total amount of the active ingredient to be administered may generally range from, for example, about 0.001 mg/kg to about 200 mg/kg, or from about 0.01 mg/kg to about 200 mg/kg body weight per day. A unit dosage may contain from, for example, about 0.05 mg to about 1500 mg of active ingredient, and may be administered one or more times per day. The daily dosage for administration by injection, including intravenous, intramuscular, subcutaneous, and parenteral injections, and use of infusion techniques may be from, for example, about 0.01 to about 200 mg/kg. The daily rectal dosage regimen may be from, for example, about 0.01 to about 200 mg/kg of total body weight. The transdermal concentration may be that required to maintain a daily dose of from, for example, about 0.01 to about 200 mg/kg.

Of course, the specific initial and continuing dosage regimen for each patient will vary according to the nature and severity of the condition as determined by the attending diagnostician, the activity of the specific peptide employed, the age of the patient, the diet of the patient, time of administration, route of administration, rate of excretion of the drug, drug combinations, and the like. The desired mode of treatment and number of doses of a peptide of the present invention may be ascertained by those skilled in the art using conventional treatment tests.

The peptides of this invention may be utilized to achieve the desired pharmacological effect by administration to a subject in need thereof in an appropriately formulated pharmaceutical composition. A subject, for example, may be a mammal, including a human, in need of treatment for a particular condition or disease. Therefore, the present invention includes pharmaceutical compositions which are comprised of a pharmaceutically acceptable carrier and a pharmaceutically effective amount of a peptide of the present invention. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A pharmaceutically effective amount of a peptide is that amount which produces a result or exerts an influence on the particular condition being treated. The peptides of the present invention may be administered with a pharmaceutically-acceptable carrier using any effective conventional dosage unit forms, including, for example, immediate and timed release preparations, orally, parenterally, topically, or the like.

For oral administration, the peptides may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the peptides of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Syrups and elixirs may be formulated with sweetening agents such as, for example, glycerol, propylene glycol, sorbitol, or sucrose. Such formulations may also contain a demulcent, and preservative, flavoring and coloring agents.

The peptides of this invention may also be administered parenterally, that is, subcutaneously, intravenously, intramuscularly, or interperitoneally, as injectable dosages of the peptide in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the peptides of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

The peptides identified by the methods described herein may be administered as the sole pharmaceutical agent or in combination with one or more other pharmaceutical agents where the combination causes no unacceptable adverse effects. For example, the peptides of this invention can be combined with known anti-obesity, or with known antidiabetic or other indication agents, and the like, as well as with admixtures and combinations thereof.

The peptides identified by the methods described herein may also be utilized, in free base form or in compositions, in research and diagnostics, or as analytical reference standards, and the like. Therefore, the present invention includes compositions which are comprised of an inert carrier and an effective amount of a peptide of the present invention. An inert carrier is any material which does not interact with the peptide to be carried and which lends support, means of conveyance, bulk, traceable material, and the like to the peptide to be carried. An effective amount of peptide is that amount which produces a result or exerts an influence on the particular procedure being performed.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000)

The peptides described herein may also be utilized, in compositions, in research and diagnostics, or as analytical reference standards, and the like. Therefore, the present invention includes compositions which are comprised of an inert carrier and an effective amount of a peptide of the present invention. An inert carrier is any material which does not interact with the peptide to be carried and which lends support, means of conveyance, bulk, traceable material, and the like to the peptide to be carried. An effective amount of peptide is that amount which produces a result or exerts an influence on the particular procedure being performed.

Peptides are known to undergo hydrolysis, deamidation, oxidation, racemization and isomerization in aqueous and non-aqueous environment. Degradation such as hydrolysis, deamidation or oxidation can readily detected by capillary electrophoresis. Enzymatic degradation notwithstanding, peptides having a prolonged plasma half-life, or biological resident time, should, at minimum, be stable in aqueous solution. For example, a peptide exhibits less than 10% degradation over a period of one day at body temperature or less than 5% degradation over a period of one day at body temperature. Stability (i.e., less than a few percent of degradation) over a period of weeks at body temperature will allow less frequent dosing. Stability in the magnitude of years at refrigeration temperature will allow the manufacturer to present a liquid formulation, thus avoid the inconvenience of reconstitution. Additionally, stability in organic solvent would provide peptide be formulated into novel dosage forms such as implant.

The structures, materials, compositions, and methods described herein are intended to be representative examples of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

The following examples are presented to illustrate the invention described herein, but should not be construed as limiting the scope of the invention in any way.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1

### Peptide Synthesis

Peptides were synthesized with an Applied Biosystems 430A peptide synthesizer using FMOC chemistry with HBTU activation on Rink amide resin. Standard Applied Biosystems protocols were used. The peptides were cleaved with 84.6% TFA, 4.4% phenol, 4.4% water, 4.4% thioanisol, and 2.2% ethanedithiol. Peptides were precipitated from the cleavage cocktail using cold tertbutylmethyl ether. The precipitate was washed with the cold ether and dried under argon. Peptides were purified with by reversed phase C₁₈ HPLC with linear water/acetonitrile gradients containing 0.1% TFA. Peptide identity was confirmed with MALDI and electrospray mass spectrometry and with amino acid analysis.

### Example 2

### Methods for Adding N-Terminal Modifying Compound

Peptides were synthesized with an Applied Biosystems 430A peptide synthesizer using FMOC chemistry with HBTU activation on Rink amide resin. Standard Applied Biosystems protocols were used. The N-terminal modifying compounds were coupled to the peptide using the same method as would be used for amino acid coupling. N-terminal modifying compounds were commercially available. In the case of amine and mercapto containing N-terminal modifying compounds, the amine and mercapto groups were protected with FMOC or trityl, respectively, during coupling to the peptide. The peptide was cleaved with 84.6% TFA, 4.4% phenol, 4.4% water, 4.4% thioanisol, and 2.2% ethanedithiol. Peptides were precipitated from the cleavage cocktail using cold tertbutylmethyl ether. The precipitate was washed with the cold ether and dried under argon. Peptides were purified with by reversed phase C₁₈ HPLC with linear water/acetonitrile gradients containing 0.1% TFA. Peptide identity was confirmed with MALDI and electrospray mass spectrometry and with amino acid analysis.

### Example 3

### Preparation of Pegylated Peptides

PEG derivatives were prepared by incubating mPEG-MAL (Nektar) with target peptides at pH 8 and room temperature using methods known to those skilled in the art. Underivatized peptides were purified from the PEGylated peptide with Cl₈ HPLC as described in Example 1.

### Example 4

### Preparation of Membranes from Cells Expressing NPY Receptor Subtypes

Human neuroblastoma KAN-TS (NPY2) cells were grown in Dulbecco's modified Eagle's medium supplemented with 2 mM glutamine, 10% fetal bovine serum, and antibiotic/antimycotic (Gibco). Human neuroblastoma SK-N-MC (NPY1) cells were grown in Dulbecco's modified Eagle's medium supplemented with 2 mM glutamine, 1 mM sodium pyruvate, 10% fetal bovine serum, and antibiotic/antimycotic (Gibco), and human NPY5 recombinant cell line (HEK-293) was grown in Dulbecco's modified Eagle's medium supplemented with 2 mM glutamine, 10% fetal bovine serum, antibiotic/antimycotic (Gibco) and 350 μg/ml G-418. All cell lines were maintained at 37° C. with 5% CO₂ in a humidified atmosphere. At 80-90% confluency, cells were harvested for membrane preparation. Cells were washed twice with 20 ml ice-cold PBS, scraped, and centrifuged for 5 minutes at 500 rpm (Beckman). Cell pellets were then homogenized in 25 mM Tris-HCl/5 mM EDTA, pH 7.7 for 2×10 seconds (Polytron 12 mm probe, 7000-8000 rpm) and centrifuged at 4° C. for 5 minutes (Beckman). The supernatant was then centrifuged at 30,000×g for 30 minutes at 4° C., and the resulting pellet was stored at −80° C. Protein concentration was measured using the Bradford Assay (BioRad), with bovine IgG as the standard.

### Example 5

### [¹²⁵I]PYY SPA Binding Assay for NPY2 Using KAN-TS Membranes

All peptides, membrane, radioligand, and beads were diluted with binding buffer (50 mM HEPES, 10 mM CaCl₂, 5 mM MgCl₂, pH 7.4 and supplemented with 0.1% bovine serum albumin). For competition assays, increasing concentrations of peptide were incubated with 50 pM human PYY (NEX-341), 200 μg wheatgerm agglutinin beads (Amersham RPNQ0001), and membrane (10 μg) in a final volume of 200 μl in a 96-well PETG plate (Perkin Elmer Life Sciences). Nonspecific binding was defined by 1 μM PYY (American Peptide, CA). Plates were incubated for 30 minutes at room temperature while shaking, removed from shaker and kept at room temperature for an additional 2.5 hours. Signal was stable for at least 3 to 18 hours. The amount of radioactivity in the samples was then quantified with a Microbeta (Perkin Elmer Life Sciences). Binding data were analyzed with PRISM (Graphpad).

### Example 6

### NPY2 Functional Activity Assessed with [³⁵S]GTPγ[S] Binding Assay

All peptides, membrane, radioligand, and beads were diluted with binding buffer (50 mM HEPES, 100 mM NaCl, 1 mM MgCl₂, 1 μM GDP, 10 μg/ml saponin, and supplemented with 0.1% BSA, pH 7.4). KAN-TS membranes (10 μg), increasing concentrations of peptide, 300 μg wheatgerm agglutinin beads (Amersham RPNQ0001), and 100 μM [³⁵S]GTPγ[S] (NEG030H) were incubated in a final volume of 100 μl in a 96-well PETG plate (Perkin Elmer Life Sciences). Nonspecific binding was determined using GTPγS (10 μM). The 96-well plates were placed on a shaker for 60 minutes at room temperature, centrifuged for 5 minutes at 2000 rpm (Beckman), and counted one hour later in a Microbeta (Perkin Elmer Life Sciences). Binding data were analyzed with PRISM (Graphpad).

### Example 7

### [¹²⁵I]PYY Binding Assay for NPY1 Using SK-N-MC Membrane

All peptides, membrane, and radioligand, and beads were diluted with binding buffer (20 mM HEPES, 137 mM NaCl, 5.4 mM KCl, 0.44 mM KH₂PO₄, 1.26 mM CaCl₂, 0.81 mM MgSO₄, pH 7.4, and supplemented with 0.3% bovine serum albumin). For competition assays, increasing concentrations of peptide were incubated with 75 pM [¹²⁵I]human PYY (NEX-341), and membrane (20-30 μg) in a final volume of 200 μl in a 96-well polypropylene plate (Perkin Elmer Life Sciences). Nonspecific binding was defined by 1 μM PYY. Plates were incubated for 2 hours at room temperature while shaking. Following the incubation, total content of the wells were transferred to Millipore HV plates (pretreated with 0.2% BSA and aspirated prior to transfer), rapidly filtered, and wash ×3 with 200 μl ice-cold binding buffer. Filters were then air-dried, 15-20 μl scintillant added (Microscint O, Packard), covered with adhesive film, and counted in a Microbeta (Perkin Elmer Life Sciences). Binding data was analyzed with PRISM (Graphpad).

### Example 8

### [¹²⁵I]PYY Binding Assay for NPY5 Using Recombinant HEK 293 Membranes

All peptides, membrane, and radioligand, and beads were diluted with binding buffer (25 mM Tris, 120 mM NaCl, 5 mM KCl, 1.2 mM KH₂PO₄, 2.5 mM CaCl₂, 1.2 mM MgSO₄, pH 7.4, and supplemented with 0.1% bovine serum albumin). For competition assays, increasing concentrations of peptide were incubated with 75 pM [¹²⁵I]human PYY (NEX-341), and membrane (10 μg) in a final volume of 200 μl in a 96-well polypropylene plate (Perkin Elmer Life Sciences). Non-specific binding was defined by 1 μM PYY. Plates were incubated for 2 hours at room temperature while shaking. Following the incubation, total content of the wells were transferred to Millipore HV plates (pretreated with 0.2% BSA and aspirated prior to transfer), rapidly filtered, and wash ×3 with 200 μl ice-cold binding buffer. Filters were then air-dried, 15-20 μl scintillant added (Microscint O, Packard), covered with adhesive film, and counted in a Microbeta (Perkin Elmer Life Sciences). Binding data was analyzed with PRISM (Graphpad).

### Example 9

### Polyclonal Antibody Production

Synthesis of the peptide Ac—CRHYLNLVTRQRY—NH₂ (SEQ ID NO: 6) was performed as described in Example 1. Peptide identity was confirmed with MALDI mass spectrometry using a PerSeptive V Biosystems Voyager DE Pro MALDI mass spectrometer. The cysteine residue was coupled to KLH using the Pierce Imject Maleimide Activated mcKLH kit and protocol (Pierce, Rockford, Ill.). Rabbits were immunized and antibodies isolated using procedures known to those of skilled in the art.

The antibodies produced in rabbits to the peptide Ac—CRHYLNLVTRQRY—NH₂ (SEQ ID NO: 6) recognized the peptides PYY(3-36) and N-terminally modified PYY(25-36) peptides, as confirmed with the enzyme-linked immunoadsorbent assay (ELISA) using methods known to those of skilled in the art.

### Example 10

### Evaluation of a Peptide's Efficacy on the Reduction of Food Intake in Lean Overnight Fasted Mice

### Fasted-Refed Acute Feeding Assay

The purpose of this protocol is to determine the effect of a single dose of a peptide on food consumption of lean overnight fasted mice. The fasted-refed mouse model is frequently used in the field of obesity to identify compounds with potential for anorectic effects. This animal model has been successfully used in the identification and characterization of the efficacy profile of compounds that are or have been used in the management of body weight in obese humans (see, e.g., Balvet, et al., Gen. Pharmacol. 13:293-297, 1982; Grignaschl, et al., Br. J. Pharmacol. 127:1190-1194, 1999; McTavish and Heel, Drug 43:713-733, 1992; Rowland, et al., Life Sci. 36:2295-2300, 1985).

A typical study includes 100-140 male mice (2 mice/cage; n=10/treatment group) with an average body weight of approximately 22 g. Mice are kept in standard animal rooms under controlled temperature and humidity and a 12/12 light dark cycle. Mice are single-housed in suspended cages with a mesh floor. Water and food are continuously available unless the animals are being fasted for the study.

The mice are fasted overnight during the dark phase (total of approx. 16-18 hr). The animal is treated with an assigned dose of peptide. Thirty minutes after dosing, pre-weighed food jars are returned to the cage. Food intake is recorded 1, 2, 4, and 24 hours post-food return. At each time point, spillage is returned to the food jar and then the food jars are weighed. The amount of food consumed is determined for each time point. Difference between treatment group is determined using appropriate statistical analysis.

### Example 11

### Evaluation of a Peptide's Efficacy on the Reduction of Body Weight and Food and Water Consumption in Obese Zucker fa/fa Rats

### Chronic Feeding Assay

The purpose of this protocol is to determine the effect of chronic administration of a peptide on body weight and food and water consumption in obese Zucker fa/fa rats. Obese Zucker fa/fa rats are frequently used in the determination of compound efficacy in the reduction of body weight. This animal model has been successfully used in the identification and characterization of the efficacy profile of compounds that are or have been used in the management of body weight in obese humans (see, e.g., Al-Barazanji, et al., Obes Res. 8:317-323, 2000; Assimacopoulos-Jeannet, et al., Am. J. Physiol. 260(2 Pt 2):R278-283, 1991; Dryden, et al., Horm. Metab. Res. 31:363-366, 1999; Edwards and Stevens, Pharmacol. Biochem. Behav. 47:865-872, 1994; Grinker, et al., Pharmacol. Biochem. Behav. 12:265-275, 1980).

A typical study includes 60-80 male Zucker fa/fa (n=10/treatment group) with an average body weight of approximately 550 g. Rats are kept in standard animal rooms under controlled temperature and humidity and a 12/12 light dark cycle. Water and food are continuously available. Rats are single-housed in large rat shoeboxes containing grid floor. Animals are adapted to the grid floors and sham-dosed with study vehicle for at least four days before the recording of two-days baseline measurement of body weight and 24-hr food and water consumption. Rats are assigned to one of 6-8 treatment groups based upon their body weight on baseline. The groups are set up so that the mean and standard error of the mean of body weight were similar.

Animals are orally gavaged daily before the dark phase of the LD/cycle for a pre-determined number of days (typically 6-14 days) with their assigned dose of peptide. At this time, body weight, food and water consumption are measured. On the final day, animals are euthanized by CO₂ inhalation, and the body weight is measured.

The efficacy of peptides of this invention on the reduction or control of body weight may be determined by using this chronic feeding assay.

All publications and patents mentioned in the above specification are incorporated herein by reference. Various modifications and variations of the described compositions and methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

1. A peptide of Formula (I)

| | |
|---|---|
| Z-RHYLNLVTRQRY-NH₂ | (SEQ ID NO: 1) |
| (I) | |

wherein
Z is selected from

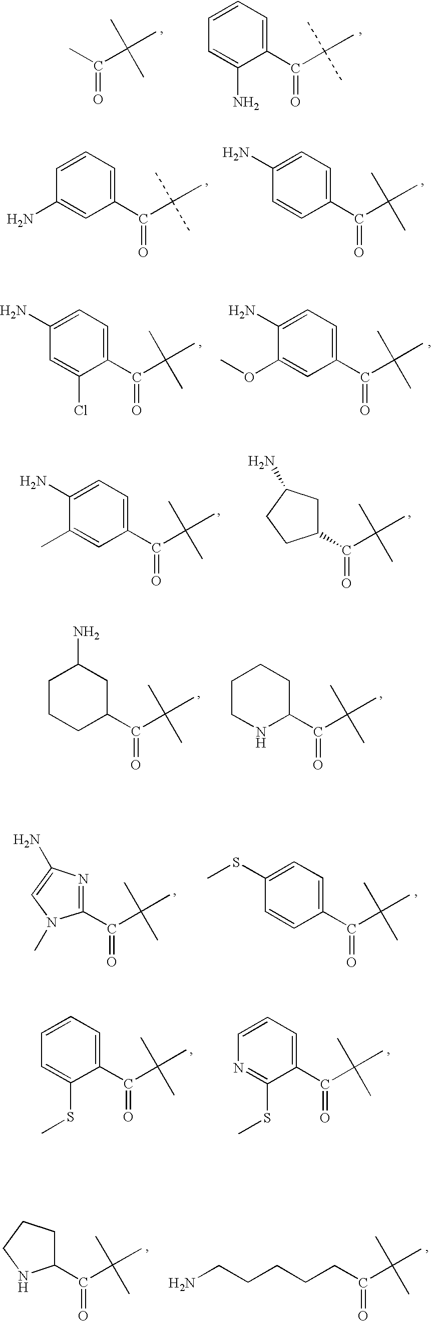

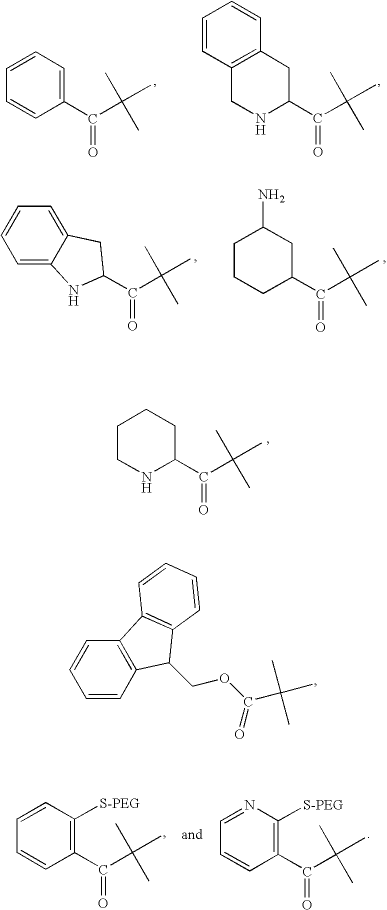

2. A peptide of Formula (II)

| | |
|---|---|
| Z-HYLNLVTRQRY-NH₂ | (SEQ ID NO:2) |
| (II) | |

wherein
Z is selected from

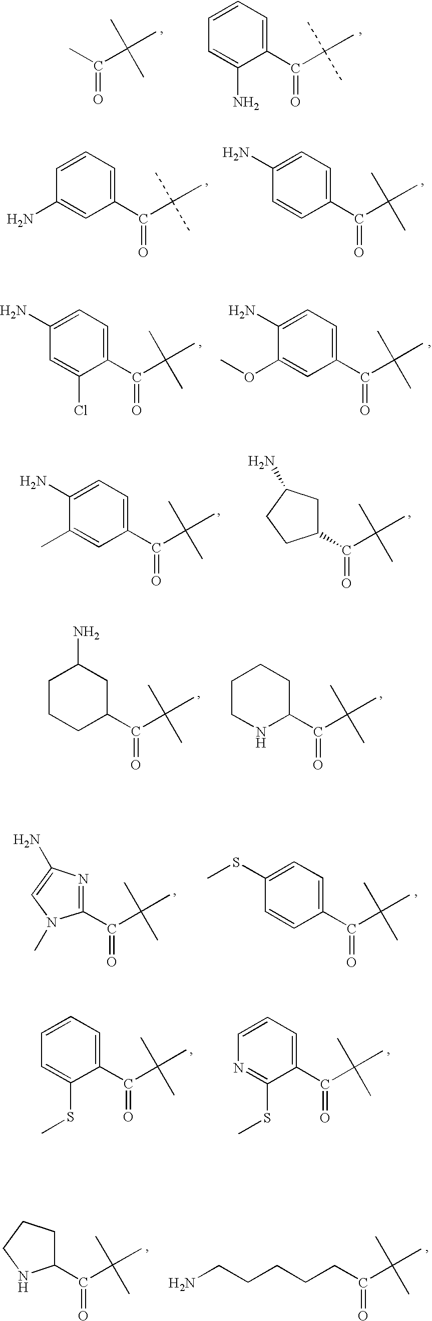

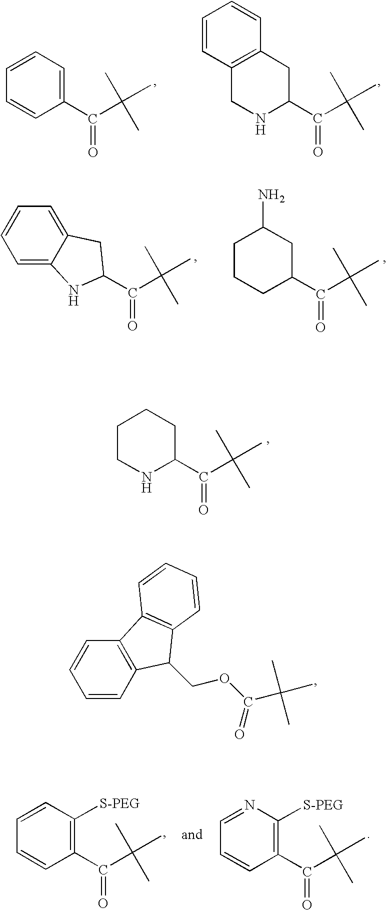

3. A peptide of Formula (III)

| | |
|---|---|
| Z-YLNLVTRQRY-NH₂ | (SEQ ID NO:3) |
| (III) | |

wherein
Z is selected from

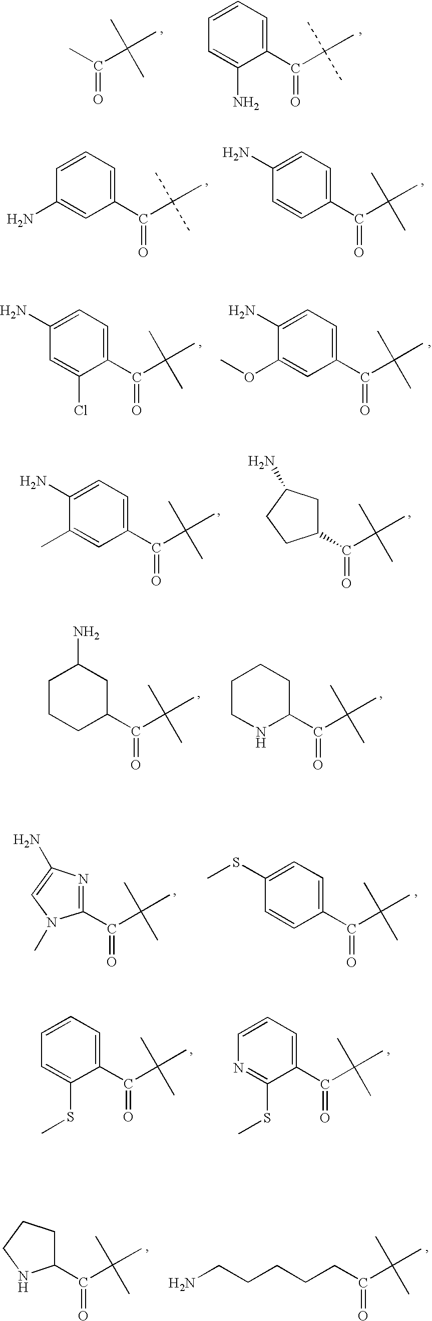

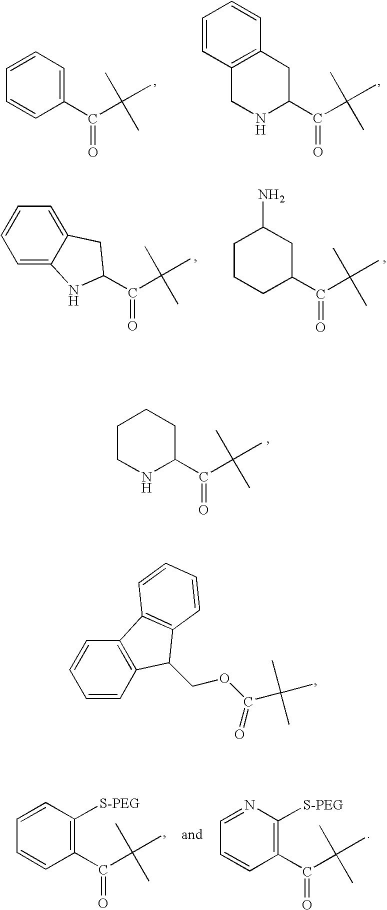

4. A peptide of Formula (IV)

| | |
|---|---|
| Z-LNLVTRQRY-NH₂ | (SEQ ID NO:4) |
| (IV) | |

wherein
Z is selected from

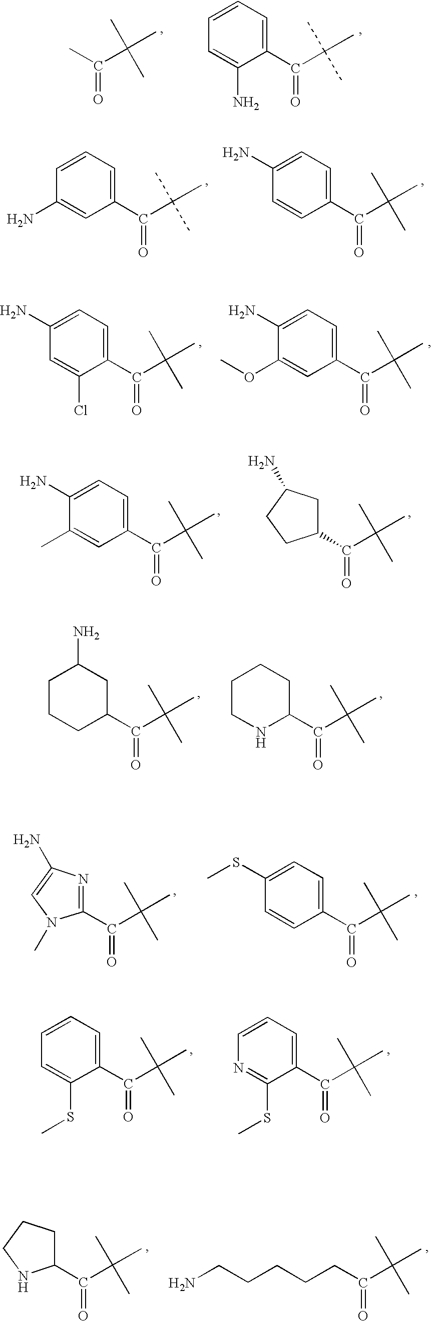

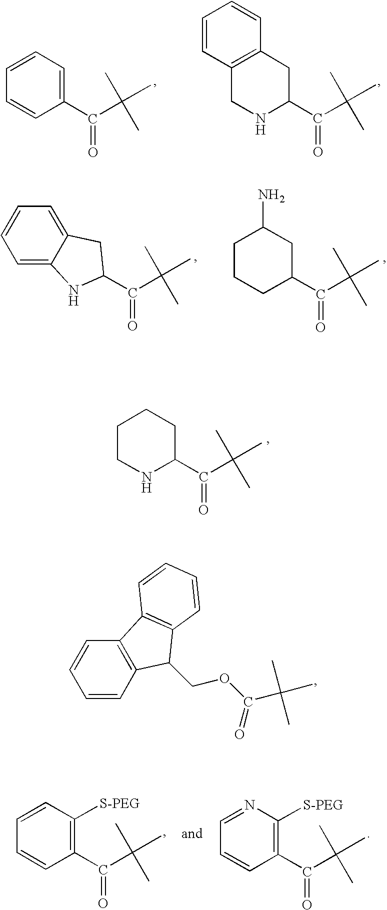

5. A peptide of Formula (V)

| | |
|---|---|
| Z-NLVTRQRY-NH₂ | (SEQ ID NO:5) |
| (V) | |

wherein
Z is selected from

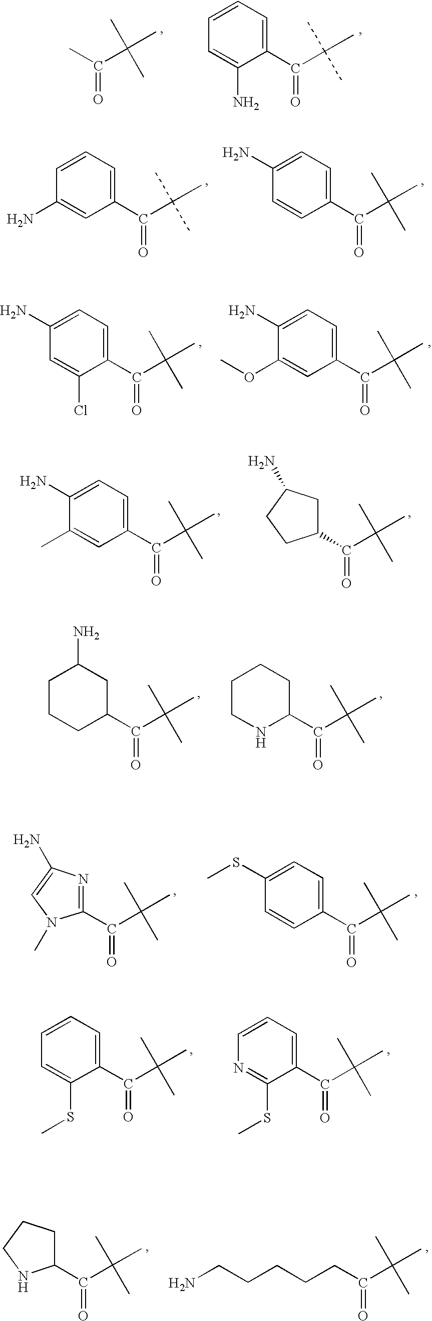

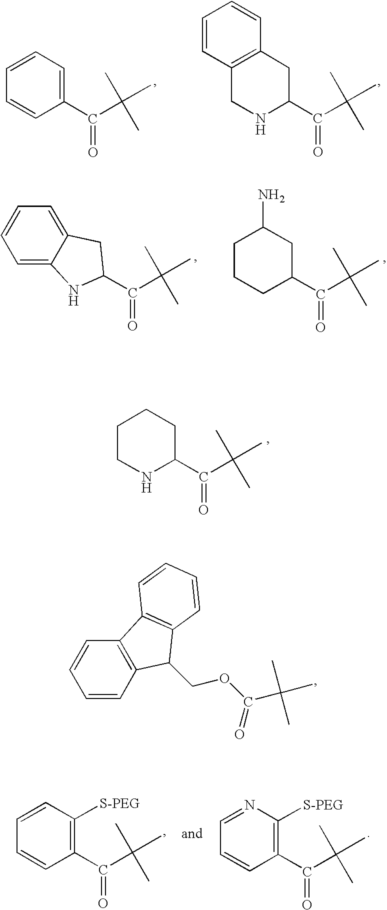

6. The peptide of any one of claims 1 to 5, wherein said peptide is PEGylated.

7. A pharmaceutical composition comprising a therapeutically effective amount of a peptide of any one of claims 1 to 5, in combination with a pharmaceutically acceptable carrier.

8. A pharmaceutical composition comprising a therapeutically effective amount of a peptide of any one of claims 1 to 5, in combination with a pharmaceutically acceptable carrier and one or more pharmaceutical agents.

9. The pharmaceutical composition of claim 8, wherein said pharmaceutical agent is an anti-obesity agent selected from the group consisting of β-3 agonists, CB-1 antagonists, appetite suppressants, and lipase inhibitors.

10. The pharmaceutical composition of claim 8, wherein said pharmaceutical agent is an agent for the treatment of diabetes selected from the group consisting of insulin, insulin derivatives, PPAR ligands, sulfonylurea drugs, α-glucosidase inhibitors, biguanides, PTP-1B inhibitors, DPP-IV inhibitors, 11-beta-HSD inhibitors, GLP-1 and GLP-1 derivatives, GIP and GIP derivatives, PACAP and PACAP derivatives, and secretin and secretin derivatives.

11. The pharmaceutical composition of claim 8, wherein said pharmaceutical agent is an agent for the treatment of lipid disorders selected from the group consisting of HMG-CoA inhibitors, nicotinic acid, fatty acid lowering compounds, lipid lowering drugs, ACAT inhibitors, bile sequestrants, bile acid reuptake inhibitors, microsomal triglyceride transport inhibitors, and fibric acid derivatives.

12. The pharmaceutical composition of claim 8, wherein said pharmaceutical agent is an anti-hypertensive agent selected from the group consisting of β-blockers, calcium channel blockers, diuretics, renin inhibitors, ACE inhibitors, AT-1 receptor antagonists, ET receptor antagonists, and nitrates.

13. A method of treating obesity comprising the step of administering to a subject in need thereof a therapeutically effective amount of a peptide of any one of claims 1 to 5 or a composition of claim 7.

14. A method of inducing weight loss comprising the step of administering to a subject in need thereof a therapeutically effective amount of a peptide of any one of claims 1 to 5 or a composition of claim 7.

15. A method of preventing weight gain comprising the step of administering to a subject in need thereof a therapeutically effective amount of a peptide of any one of claims 1 to 5 or a composition of claim 7.

16. A method of treating obesity-related disorders comprising the step of administering to a subject in need thereof a therapeutically effective amount of a peptide of any one of claims 1 to 5 or a composition of claim 7.

17. The method of claim 16, wherein said obesity-related disorder is selected from the group consisting of dyslipidemia, cholesterol gallstones, gallbladder disease, gout, cancer, menstrual abnormalities, infertility, polycystic ovaries, osteoarthritis, sleep apnea, hypertriglyceridemia, Syndrome X, type 2 diabetes, atherosclerotic diseases, hyperlipidemia, hypercholesteremia, low HDL levels, hypertension, cardiovascular disease, coronary heart disease, coronary artery disease, cerebrovascular disease, stroke, and peripheral vessel disease.

18. A method of treating obesity comprising the step of administering to a subject in need thereof a therapeutically effective amount of a peptide of any one of claims 1 to 5 in combination with one or more pharmaceutical agents.

19. The method of claim 18, wherein the peptide of any one of claims 1 to 5 and one or more pharmaceutical agents are administered as a single pharmaceutical dosage formulation.

20. A method of treating obesity comprising the step of administering to a subject in need thereof a therapeutically effective amount of a composition of claim 8, 9, 10, 11, or 12.

21. A method of treating obesity-related disorders comprising the step of administering to a subject in need thereof a therapeutically effective amount of a composition of claim 8, 9, 10, 11, or 12.

22. Peptides according to any one of claims 1 to 5 for the treatment and/or prophylaxis of obesity and obesity-related disorders.

23. (canceled)

24. (canceled)

25. (canceled)
